(19) United States
(12) Patent Application Publication
(10) Pub. No.: US 2012/0191199 A1
(43) Pub. Date: Jul. 26, 2012

(54) METHOD AND APPARATUS FOR DISTAL RADIOULNAR JOINT (DRUJ) ARTHROPLASTY

(76) Inventor: Michael Raemisch, North Salt Lake, UT (US)

(21) Appl. No.: 13/330,446

(22) Filed: Dec. 19, 2011

Related U.S. Application Data
(60) Provisional application No. 61/424,256, filed on Dec. 17, 2010.
(60) Provisional application No. 61/445,599, filed on Feb. 23, 2011.

(51) Int. Cl. A61F 2/30; A61B 17/56

(52) U.S. Cl. 623/18.11; 606/87

(57) ABSTRACT

Apparatus for reconstructing a joint of the sort comprising a first bone having a first articular surface and a second bone having a second articular surface, wherein the first articular surface and the second articular surface engage one another, the apparatus comprising:
a prosthesis for replacing at least a portion of the first articular surface of the first bone, the prosthesis comprising:
   a prosthesis plate for positioning against an outside surface of the first bone; and
   a prosthesis body connected to the prosthesis plate, the prosthesis body comprising a first prosthetic articular surface which generally matches the shape and size of the first articular surface.

### REFERENCE TO PENDING PRIOR PATENT APPLICATIONS

This patent application claims benefit of:

(i) pending prior U.S. Provisional Patent Application Ser. No. 61/424,256, filed Dec. 17, 2010 by Michael Raemisch for DISTAL RADIUS ULNAR JOINT IMPLANT AND SURGICAL TECHNIQUE (Attorney's Docket No. SNYDER-37 PROV); and

(ii) pending prior U.S. Provisional Patent Application Ser. No. 61/445,599, filed Feb. 23, 2011 by Michael Raemisch for DISTAL RADIOULNAR JOINT IMPLANT AND SURGICAL TECHNIQUE (Attorney's Docket No. SNYDER-42 PROV).

The two (2) above-identified patent applications are hereby incorporated herein by reference.

### FIELD OF THE INVENTION

This invention relates to surgical apparatus and procedures in general, and more particularly to surgical apparatus and procedures for distal radioulnar joint (DRUJ) arthroplasty. Even more particularly, this invention relates to replacement arthroplasty of the distal radius portion of the distal radioulnar joint and/or replacement arthroplasty of the distal ulnar portion of the distal radioulnar joint.

### BACKGROUND OF THE INVENTION

The distal radioulnar joint (DRUJ) is the joint of the wrist that links the radius and the ulna. It is the joint by which the wrist twists from a palm-up position (supination) to a palm-down position (pronation). The DRUJ consists of two articular surfaces: the medial (ulnar) side of the distal end of the radius bone at the wrist, sometimes called the “sigmoid notch”, and the mainly anterior surface of the distal end of the ulna bone at the wrist, sometimes called the “seat” of the ulna. The sigmoid notch of the radius is a shallow, slightly concave shape while the seat of the ulna is convex. The radius of curvature of the sigmoid notch is larger than that of the ulna seat. This allows for a wide range of motion and flexibility. See FIGS. 1 and 2. However, this architecture is intrinsically unstable and, therefore, the joint relies heavily on soft tissue connections for stability. Soft tissue elements critical for maintaining stability of the DRUJ include the Triangular Fibrocartilage Complex (TFCC), the Extensor Carpi Ulnaris (ECU) subsheath, the distal radio-ulna ligaments (volar and dorsal), and the volar ulnocarpal ligaments.

Like other joints, the DRUJ can be affected by arthritis, a painful deterioration of the cartilage of a joint. One method of treating painful arthritis involves replacing the arthritic joint with an artificial joint. This procedure is commonly referred to as “joint replacement arthroplasty”. In joint replacement arthroplasty, the damaged joint surface is surgically removed and replaced with an artificial joint surface.

Current DRUJ joint replacement arthroplasty devices suffer from various shortcomings. One type of implant (prosthesis) replaces the entire portion of the distal ulna, i.e., the head of the ulna. This effectively replaces the joint surface of the distal ulna. However, in doing so, the integrity of vital anatomic structures is compromised (i.e., the TFCC, the ECU subsheath, and the ligaments in the region). These structures act to stabilize the DRUJ, maintaining a mechanically appropriate relationship between the radius and ulna. The nature of the aforementioned device also precludes re-creation or reconstruction of these stabilizing anatomic structures. Left unstable, the joint risks becoming painful and/or weak. In addition, instability may lead to failure of the implant (prosthesis).

Another disadvantage facing current devices for replacing the ulnar head relates to the fixation method. These implants are typically secured by means of a long, rod-like stem, which is inserted into the interior of the tube-shaped ulna bone (i.e., into the intramedullary canal of the ulna). This can be a technically difficult procedure. In addition, it can be difficult to remove the intramedullary device should the need arise. Among other things, removal of an intramedullary device can result in significant bone destruction leaving few options for joint salvage.

The radial side of the DRUJ is commonly referred to as the “sigmoid notch”. Like the devices used for replacement arthroplasty of the distal ulna, devices for resurfacing the sigmoid notch violate native structures that maintain the stability of the DRUJ. In addition, current devices to replace the sigmoid notch cannot be used alone—they require a matching ulnar side component.

Of the various factors affecting DRUJ joint replacement arthroplasty devices, anatomic stability may be the most critical. Left unstable, the distal end of the ulna moves abnormally. This can be a cause of both pain and weakness for the patient, and is a potential cause of early mechanical failure of the device. Unfortunately, due to the nature of their design, existing DRUJ joint replacement arthroplasty devices violate key anatomic elements and result in a compromise of the stability of the joint.

One potential solution is to design an implant that incorporates more intrinsic mechanical stability to compensate for the mechanical stability sacrificed by loss of normal anatomic stabilizing structures. Ironically, however, a device that incorporates additional mechanical stability can be just as problematic. By way of example but not limitation, a mechanically over-stable device can impose increased stress on the prosthesis, or on the bone-prosthesis interface. Either of these can create pain or mechanical failure by loosening or breakage. Constraining the joint to gain stability also sacrifices flexibility, resulting in decreased range of motion in the joint.

An ideal joint replacement arthroplasty implant device is one that effectively replaces the articular surface, maintains the integrity of key anatomic structures (or allows for reconstruction of such structures when not present or compromised), and is mechanically sound. These factors are balanced by the additional consideration of technical ease in both the index procedure and subsequent procedures if required. Such a device is provided by the present invention, which will hereinafter be discussed.

In connection with the following description of the present invention, it should be appreciated that, while the invention is discussed in connection with the DRUJ, key elements of the invention are applicable to joint arthroplasty for other joints. These key elements include resection of only the articular surface, maintaining vital surrounding structures, and fixation of the implant via plate and screws fixed to the outer surface of the bone such that the fixation is remote from the articular surface. Examples of other joints where the present invention may be applicable include, but are not limited to, the capitellum of the distal humerus in the elbow, the Great Toe metatarsophalangeal joint, the joints of the hand, the ankle, etc.

### SUMMARY OF THE INVENTION

The present invention provides a new and improved method and apparatus for distal radioulnar joint (DRUJ) arthroplasty.

Among other things, the present invention provides replacement arthroplasty of the distal radius portion of the distal radioulnar joint. To this end, a novel radius prosthesis is provided for the distal radius portion of the distal radioulnar joint, and a novel radius cutting jig is provided for preparing the distal radius portion of the distal radioulnar joint to receive the radius prosthesis.

Among other things, and as will hereinafter be discussed, the present invention replaces only the articular surface of sigmoid notch of the radius. It spares critical anatomic elements attaching to the radius that function to maintain stability of the DRUJ (the radial attachment of the TFCC, and the volar and dorsal distal radioulnar ligaments). This is accomplished by the unique nature of the design. The prosthesis is fixed in place by a plate and screws remote from the articular surface. This design allows for preservation of these critical elements and for their reconstruction should the need arise. Further, the design facilitates implantation and revision.

In addition, the present invention provides replacement arthroplasty of the distal ulnar portion of the distal radioulnar joint. To this end, a novel ulnar prosthesis is provided for the distal ulnar portion of the distal radioulnar joint, and a novel ulnar cutting jig is provided for preparing the distal ulnar portion of the distal radioulnar joint to receive the ulnar prosthesis.

Among other things, and as will hereinafter be discussed, the present invention replaces only the articular surface of the ulna, rather than replacing the entire head of the ulna. Replacing only the articular surface of the ulna, rather than the entire head of the ulna, has several significant advantages. First, the ulnar head is the mechanical fulcrum of the wrist and forearm. As such, the ulnar head transfers forces from the hand to the forearm as it counteracts the gravity force acting distal to the wrist at the hand. Second, portions of the ulnar head are key soft tissue attachment sites for critical soft tissue structures that stabilize the DRUJ. These critical soft tissue structures are preserved when only the articular surface of the ulna is removed. Third, should the native soft tissue structures of the DRUJ be compromised or non-viable due to disease state, soft tissue reconstruction procedures remain feasible due to the presence of the remaining portion of the ulnar head and the remote position of the fixation method.

Significantly, the present invention accurately restores the biomechanical function of the DRUJ by maintaining the native ulnar head as the fulcrum of the forearm axis and avoiding violation of key soft tissue structures that stabilize the DRUJ. The present invention also allows for additional surgical procedures to be performed on the DRUJ if necessary. These additional surgical procedures may include soft tissue reconstruction, shortening of the ulna, replacement of the ulna, etc.

In one form of the present invention, there is provided apparatus for reconstructing a joint of the sort comprising a first bone having a first articular surface and a second bone having a second articular surface, wherein the first articular surface and the second articular surface engage one another, the apparatus comprising:

a prosthesis for replacing at least a portion of the first articular surface of the first bone, the prosthesis comprising:
a prosthesis plate for positioning against an outside surface of the first bone; and
a prosthesis body connected to the prosthesis plate, the prosthesis body comprising a first prosthetic articular surface which generally matches the shape and size of the first articular surface.

In another form of the present invention, there is provided apparatus for reconstructing a joint of the sort comprising a first bone having a first articular surface and a second bone having a second articular surface, wherein the first articular surface and the second articular surface engage one another, the apparatus comprising:

a cutting jig for preparing the first bone to receive a prosthesis, the cutting jig comprising:
a plate for positioning against the outside surface of the first bone; and
a cutting guide connected to the plate, the cutting guide comprising at least one longitudinal cutting slot.

In another form of the present invention, there is provided apparatus for reconstructing a joint of the sort comprising a first bone having a first articular surface and a second bone having a second articular surface, wherein the first articular surface and the second articular surface engage one another, the apparatus comprising:

a cutting jig for preparing the first bone to receive a prosthesis, the cutting jig comprising:
a plate for positioning against the outside surface of the first bone; and
a cutting guide connected to the plate, the cutting guide comprising a first guide surface for use in cutting a first facet on the first bone, a second guide surface for use in cutting a second facet on the first bone, and a third guide surface for use in cutting a third facet on the first bone.

In another form of the present invention, there is provided a method for reconstructing a joint of the sort comprising a first bone having a first articular surface and a second bone having a second articular surface, wherein the first articular surface and the second articular surface engage one another, the method comprising:

providing a prosthesis for replacing at least a portion of the first articular surface of the first bone, the prosthesis comprising:
a prosthesis plate for positioning against an outside surface of the first bone; and
a prosthesis body connected to the prosthesis plate, the prosthesis body comprising a first prosthetic articular surface which generally matches the shape and size of the first articular surface; and

securing the prosthesis to the first bone so that the first prosthetic articular surface is positioned in place of the first articular surface.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1 and 2 are schematic views showing the distal radioulnar joint (DRUJ);

FIGS. 3-12 are schematic views showing a novel cutting jig for preparing the distal radius portion of the distal radioulnar joint to receive the radius prosthesis;

FIGS. 13-17 are schematic views showing a novel prosthesis for the distal radius portion of the distal radioulnar joint;

FIGS. 18-25 are schematic views showing a novel cutting jig for preparing the distal ulnar portion of the distal radioulnar joint to receive the ulnar prosthesis;

FIGS. 26-29 are schematic views showing a novel prosthesis for the distal ulnar portion of the distal radioulnar joint;

FIG. 30 is a schematic view showing a total joint arthroplasty effected in accordance with the present invention;

FIGS. 31 and 32 are schematic views showing an alternative form of a cutting jig for preparing the distal ulnar portion of the distal radioulnar joint to receive the ulnar prosthesis;

FIGS. 33-41 are schematic views showing various steps in a total joint arthroplasty effected in accordance with the present invention;

FIG. 42 is a schematic view showing a radial side reconstruction effected in accordance with the present invention;

FIG. 43 is a schematic view showing an ulnar side reconstruction effected in accordance with the present invention; and

FIG. 44 is a schematic view showing an ulnar side reconstruction in combination with an osteotomy procedure.

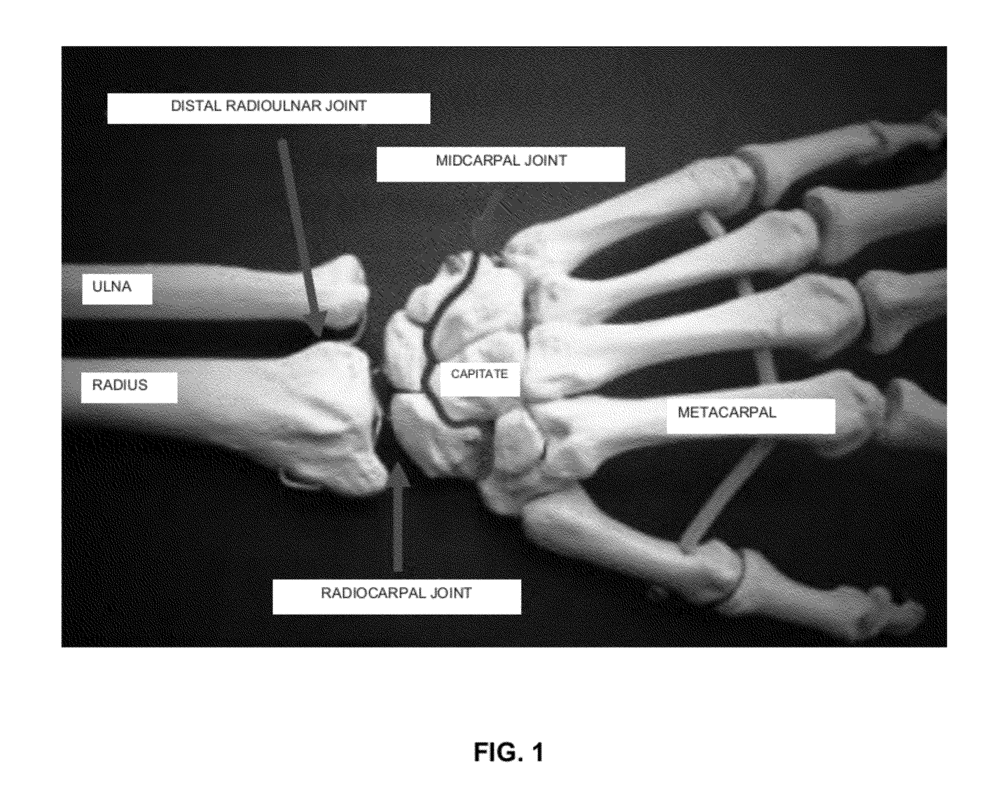

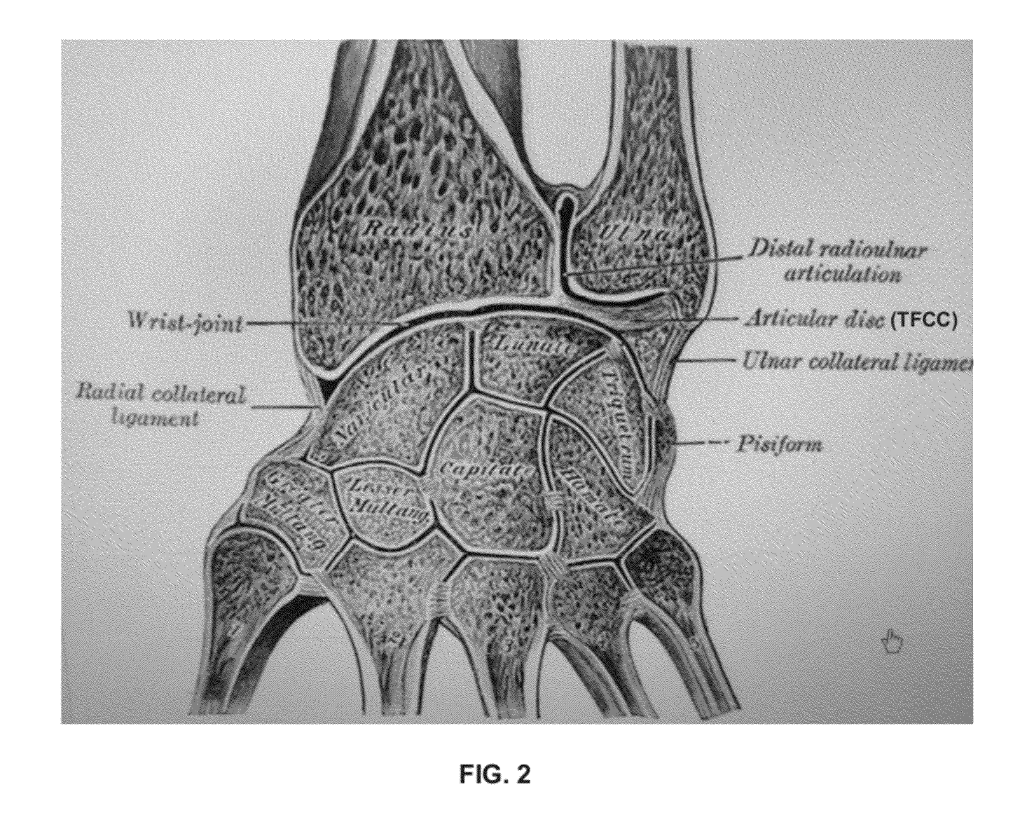

### DETAILED DESCRIPTION OF THE PREFERRED EMBODIMENTS

The present invention provides a new and improved method and apparatus for distal radioulnar joint (DRUJ) arthroplasty.

Among other things, the present invention provides replacement arthroplasty of the distal radius portion of the distal radioulnar joint. To this end, a novel prosthesis is provided for the distal radius portion of the distal radioulnar joint, and a novel cutting jig is provided for preparing the distal radius portion of the distal radioulnar joint to receive the radius prosthesis.

In addition, the present invention provides replacement arthroplasty of the distal ulnar portion of the distal radioulnar joint. To this end, a novel prosthesis is provided for the distal ulnar portion of the distal radioulnar joint, and a novel cutting jig is provided for preparing the distal ulnar portion of the distal radioulnar joint to receive the ulnar prosthesis.

### Novel Cutting Jig for Preparing the Distal Radius Portion of the Distal Radioulnar Joint to Receive the Radius Prosthesis

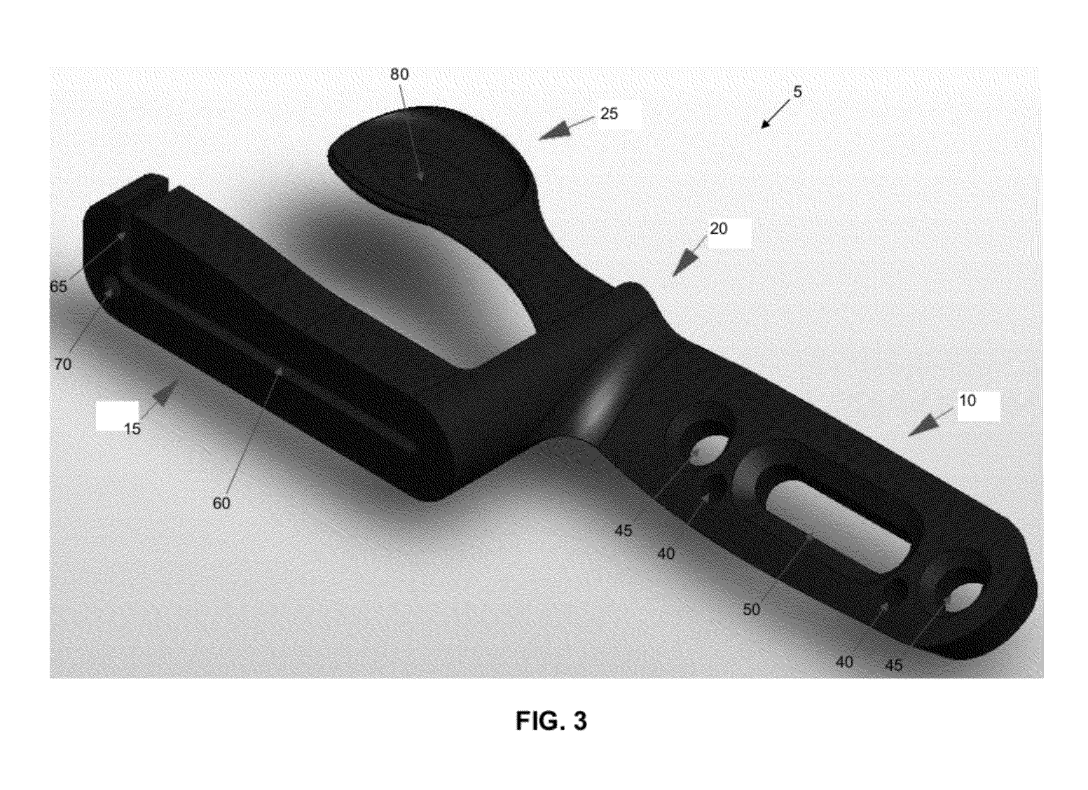

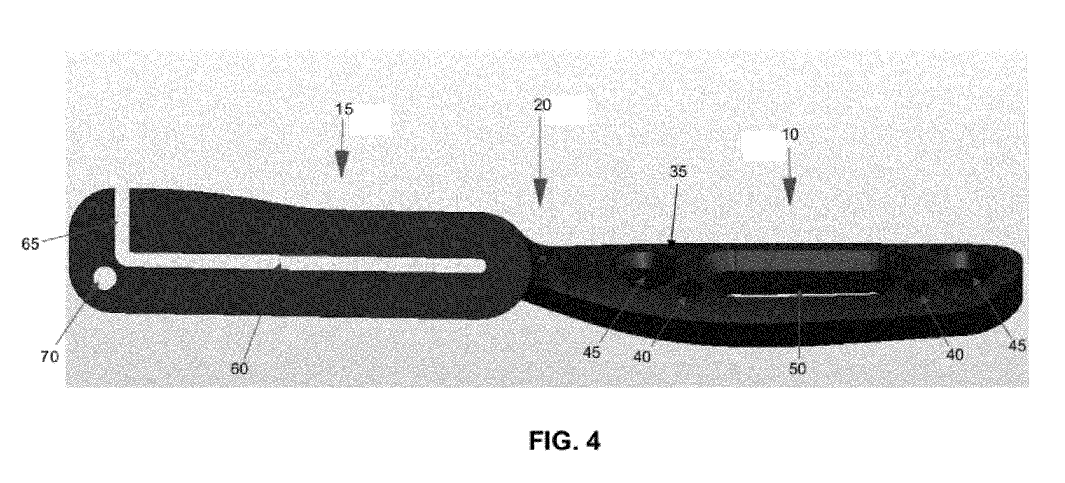

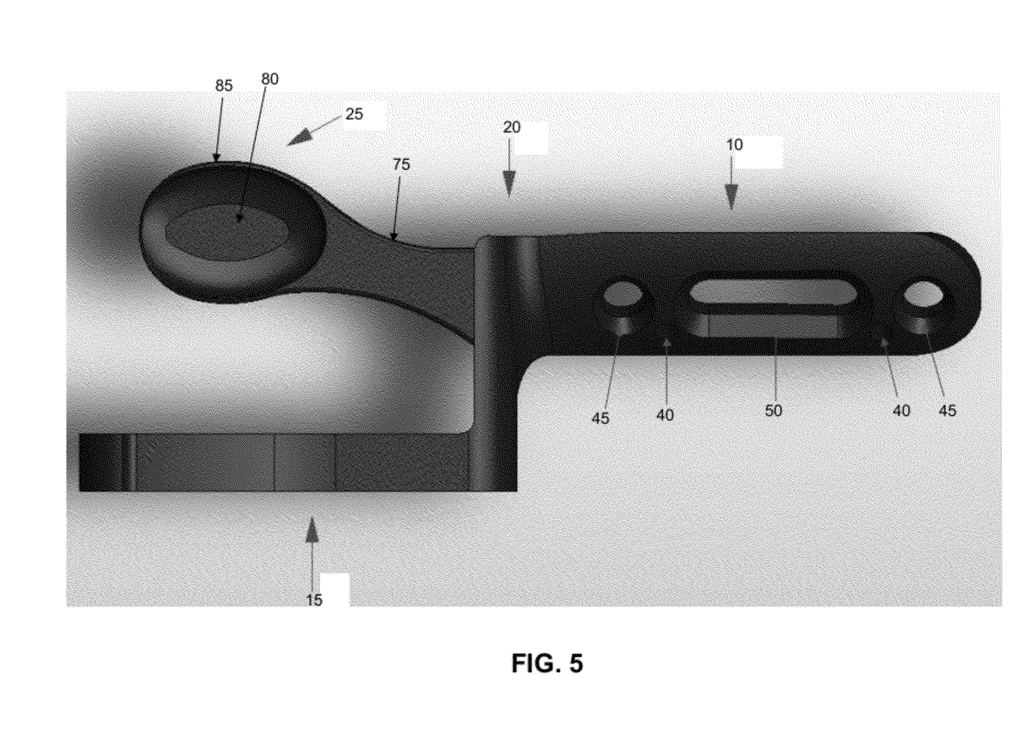

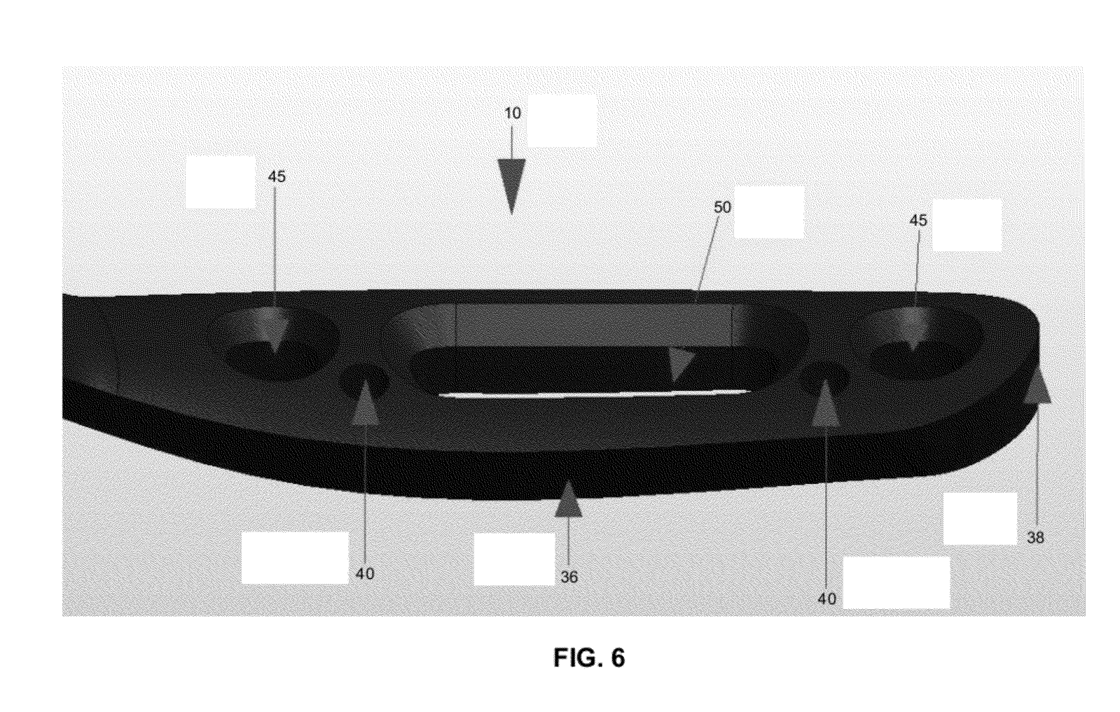

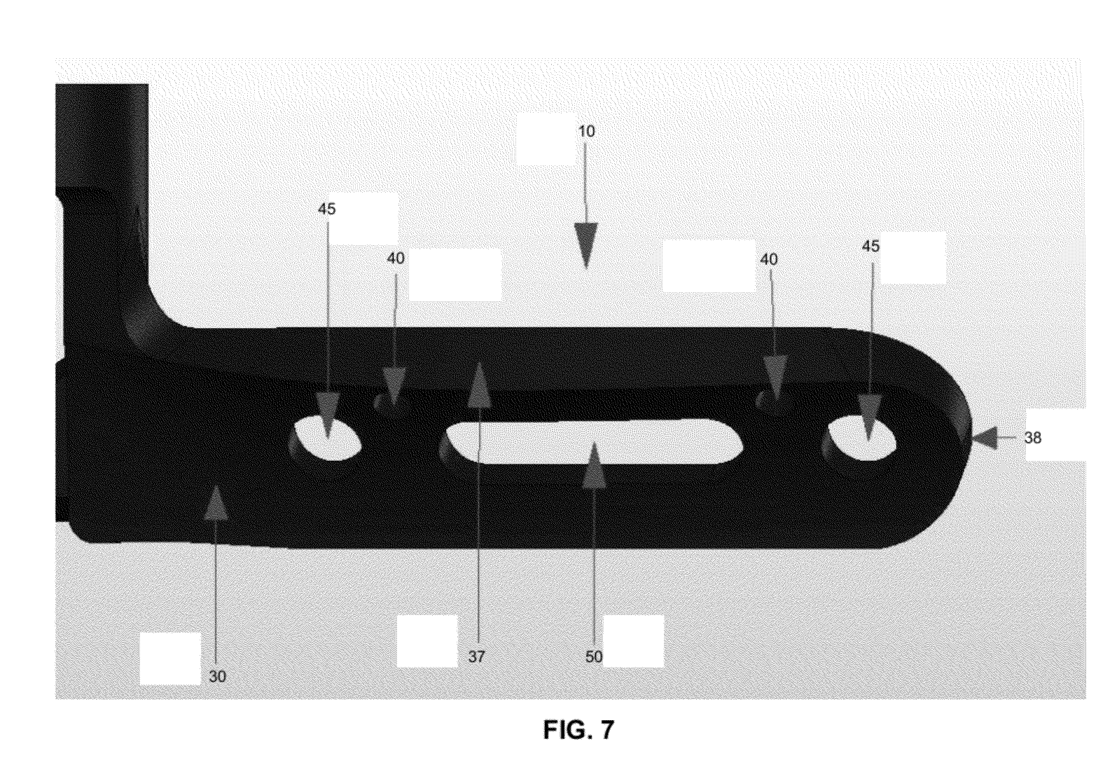

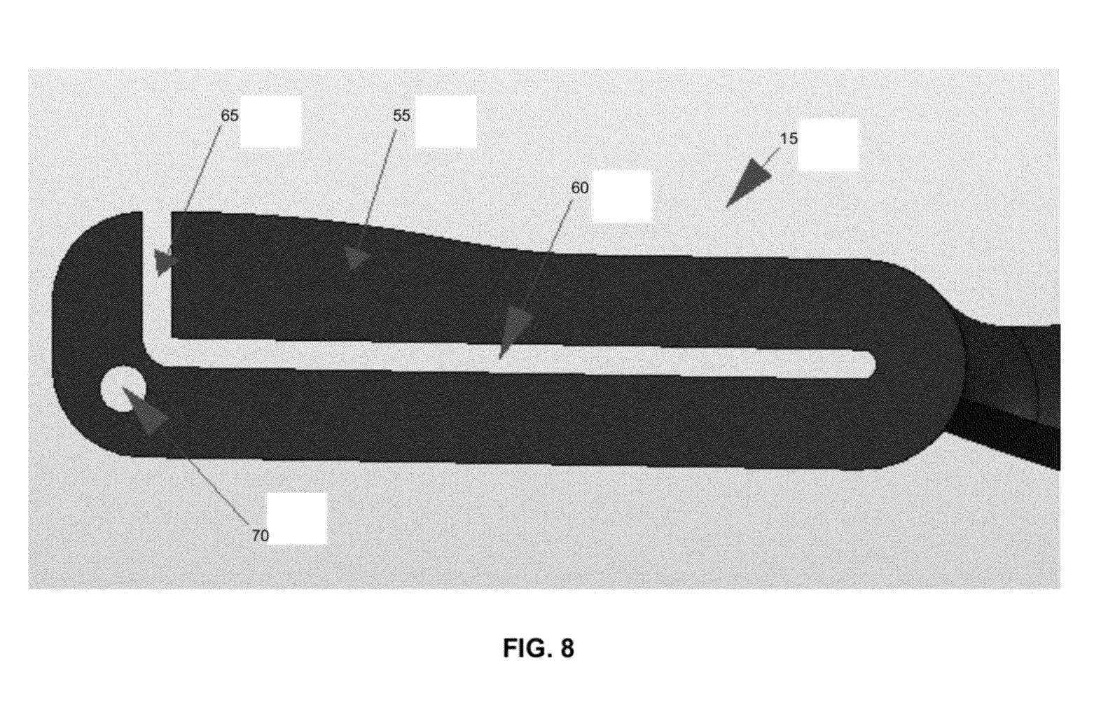

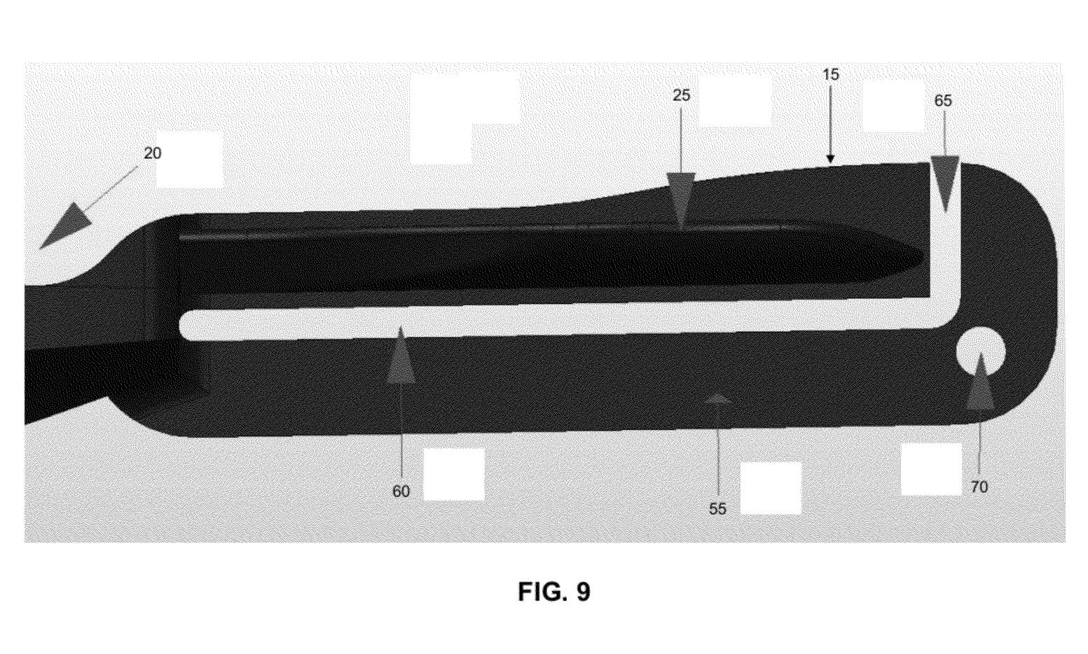

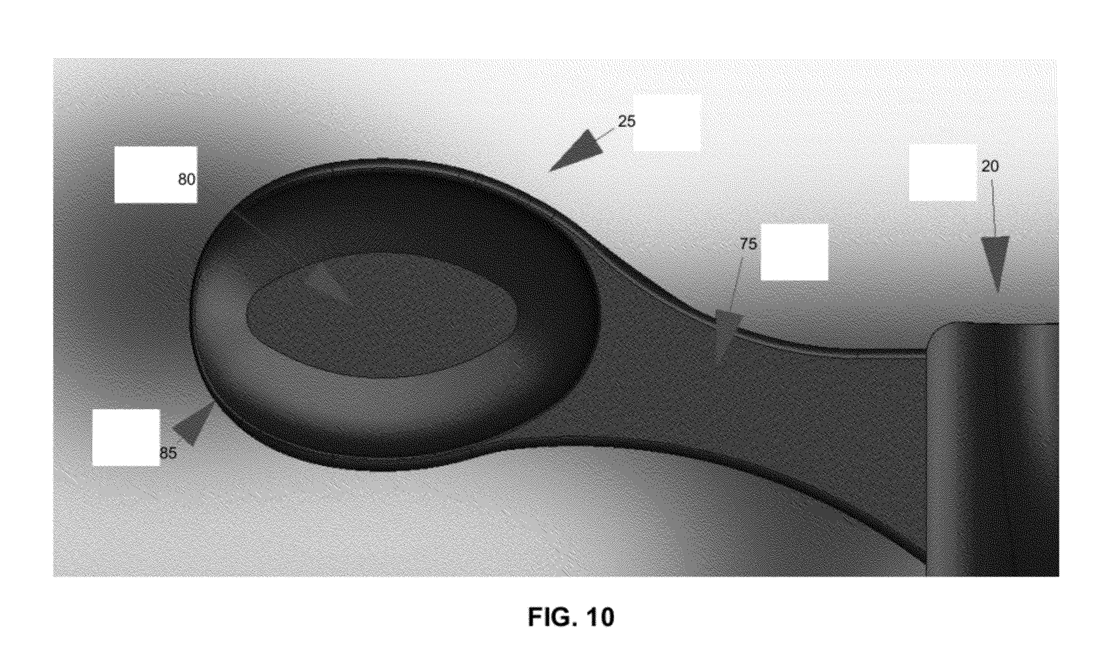

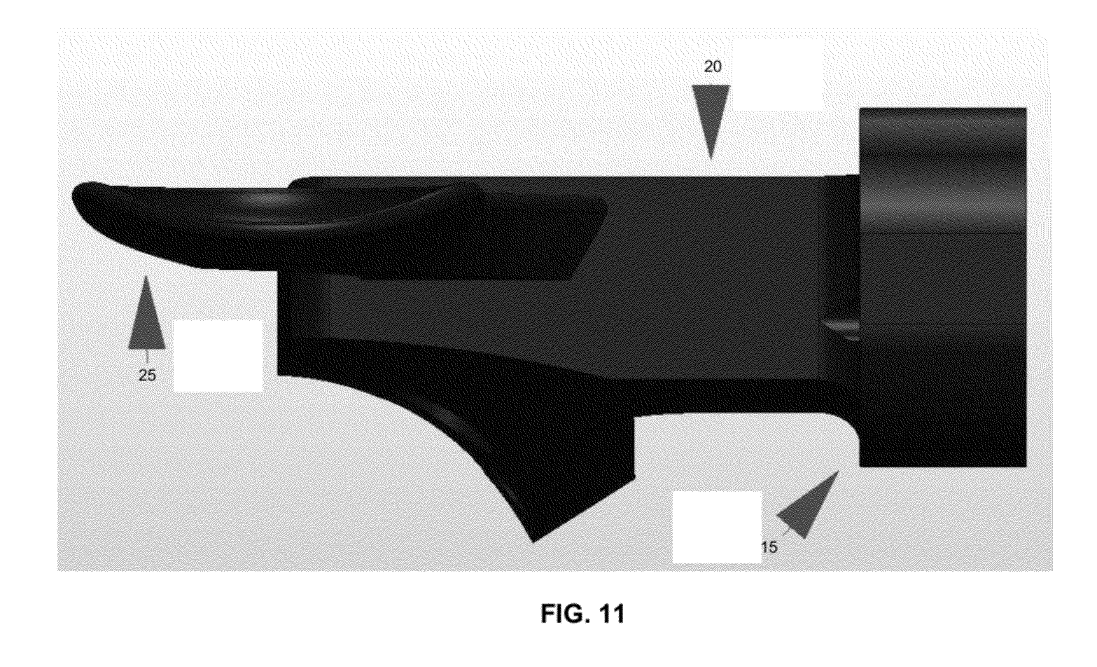

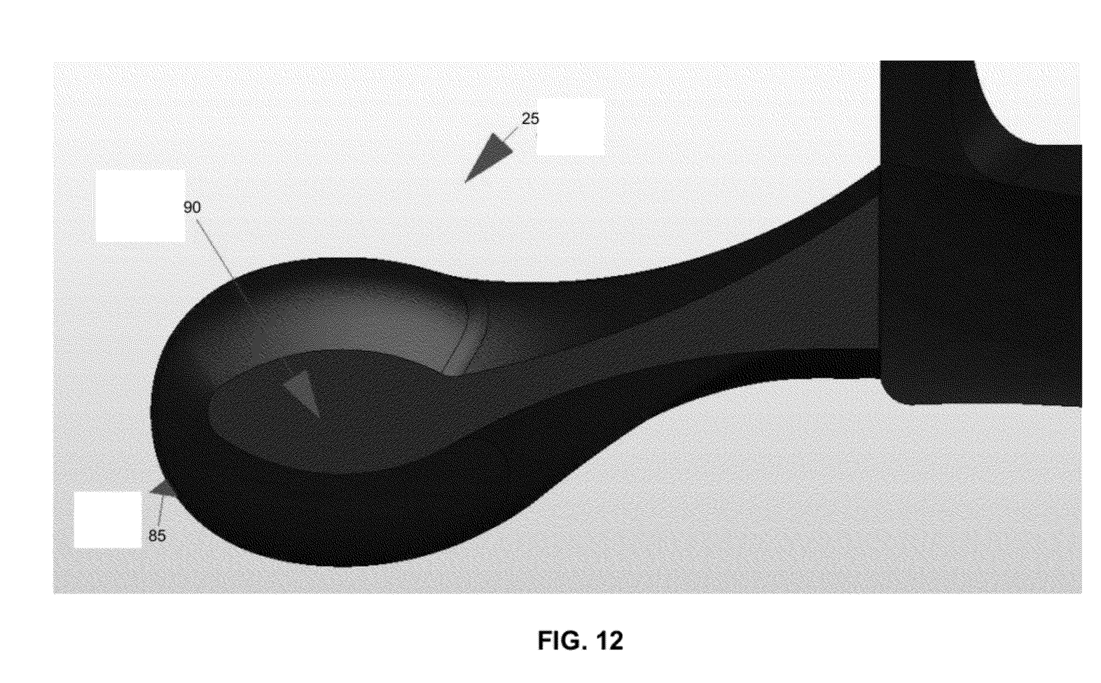

Looking first at FIGS. 3-12, there is shown a novel radius cutting jig 5 for preparing the distal radius portion of the distal radioulnar joint to receive the radius prosthesis. Radius cutting jig 5 generally comprises a plate 10 and a cutting guide 15. A neck 20 connects plate 10 to cutting guide 15. Radius cutting jig 5 preferably also comprises a spoon 25 which is also connected to plate 10 via neck 20.

Plate 10 is configured to generally match the contours of the distal radius and substantially matches the plate portion of the radius prosthesis (see below). To this end, plate 10 generally comprises a concave inner bone-engaging surface 30 and a convex outer surface 35. Plate 10 also comprises a pair of side surfaces 36, 37 and an end surface 38. In addition, plate 10 comprises two holes 40 for accommodating guidewires (see below), two or more generally circular screw holes 45, and a generally oblong screw hole 50.

Cutting guide 15 is designed to accept a sagittal saw or other cutting means of the sort well known in the art so as to enable a surgeon to safely and precisely excise a wafer or portion of compromised bone and/or articular surface at the distal end of the radius in the region of the sigmoid notch. To this end, cutting guide 15 comprises a top face 55, a longitudinal cutting slot 60 and a transverse cutting slot 65. Transverse cutting slot 65 extends at a right angle to longitudinal cutting slot 60 and communicates with longitudinal cutting slot 60, such that when cutting guide 15 is used to excise a wafer or portion of compromised bone and/or articular surface from the radius, a right angle seat will be formed in the radius, as will hereinafter be discussed. Cutting guide 15 also comprises a hole 70 on its distal end for accommodating a guidewire, as will hereinafter be discussed.

Spoon 25 of radius cutting jig 5 is designed to gently press against the ulnar head so as to distract the ulnar head and protect it while radius cutting jig 5 is used to excise a portion of the radius (see below). Spoon 25 comprises a shaft 75 which is connected to the remainder of radius cutting jig 5 by neck 20, and a basin 80 terminating in a rim 85. Spoon 25 includes a bottom surface 90 which faces the radius when radius cutting jig 5 is mounted to the radius. Applying slight pressure on the ulna when the articular surface of the ulna is mounted in spoon 25 may further stabilize radius cutting jig 5 and help the surgeon avoid damaging the ulna during the surgical excision of the surface of the sigmoid notch (see below).

### Novel Prosthesis for the Distal Radius Portion of the Distal Radioulnar Joint

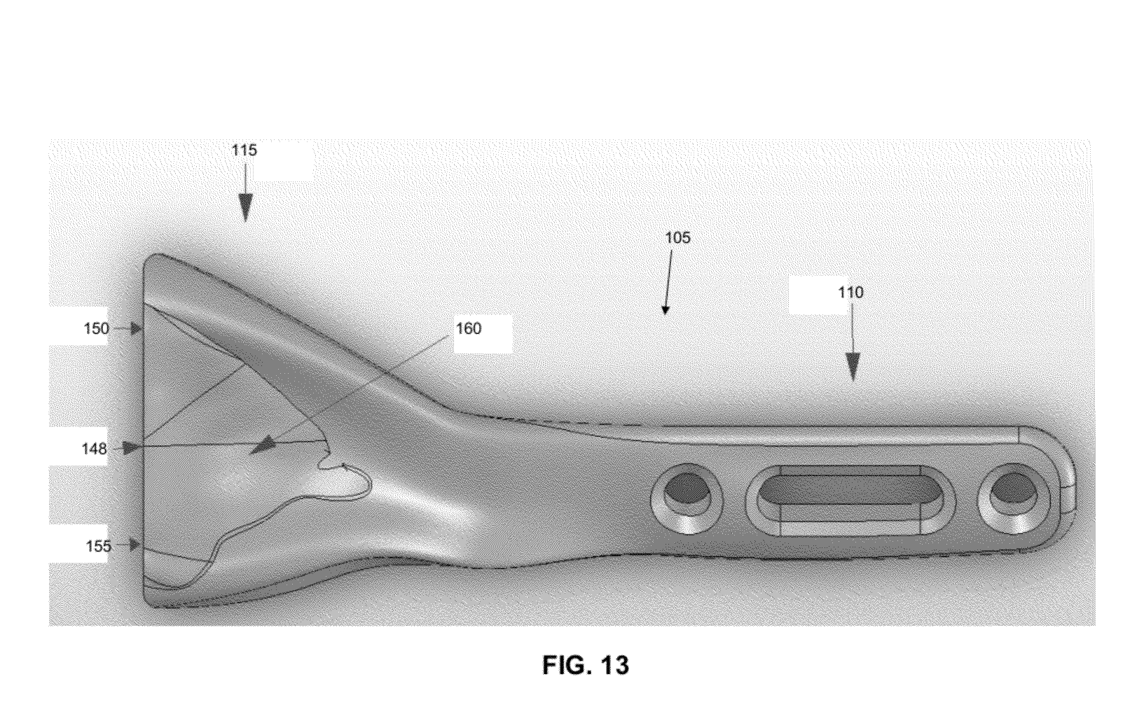

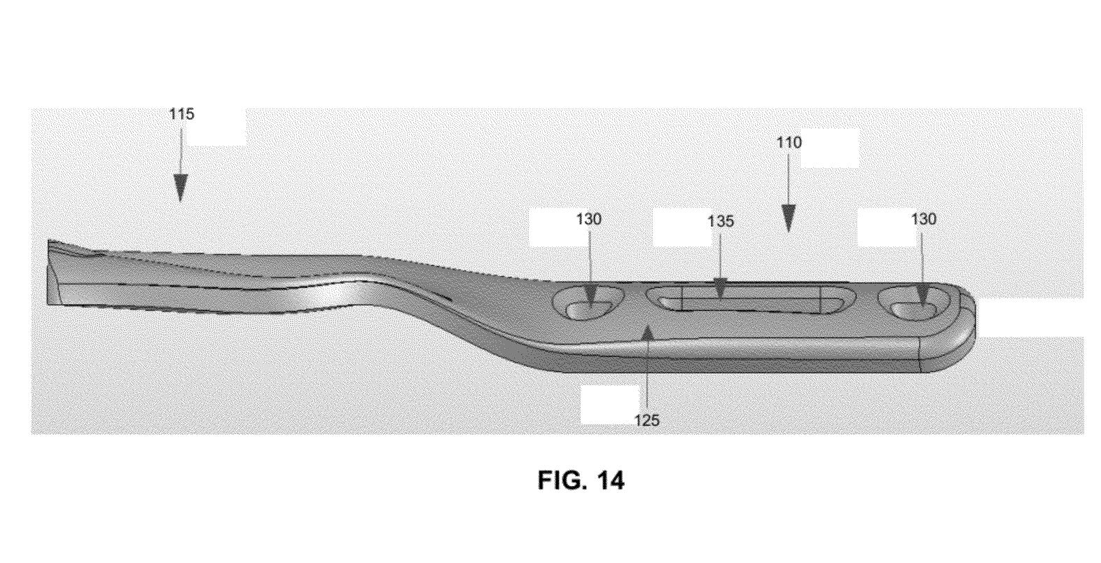

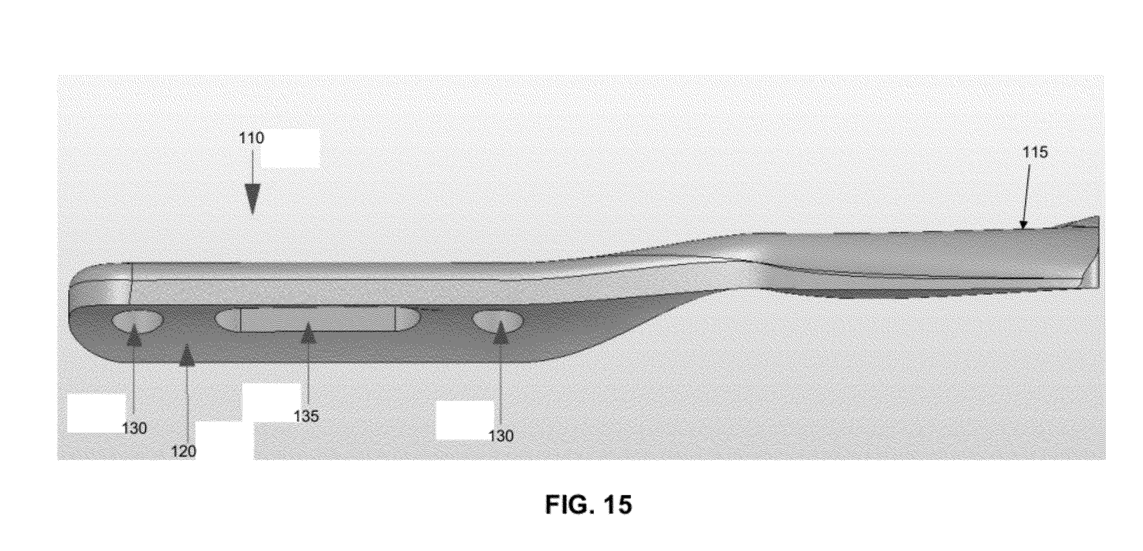

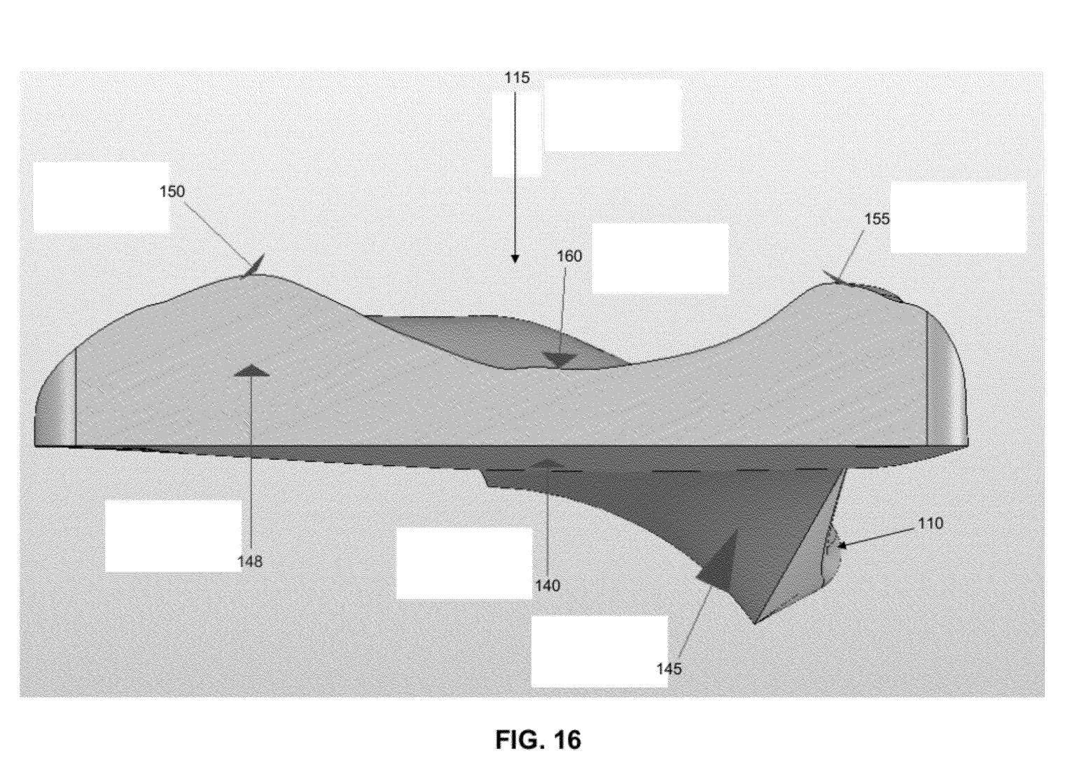

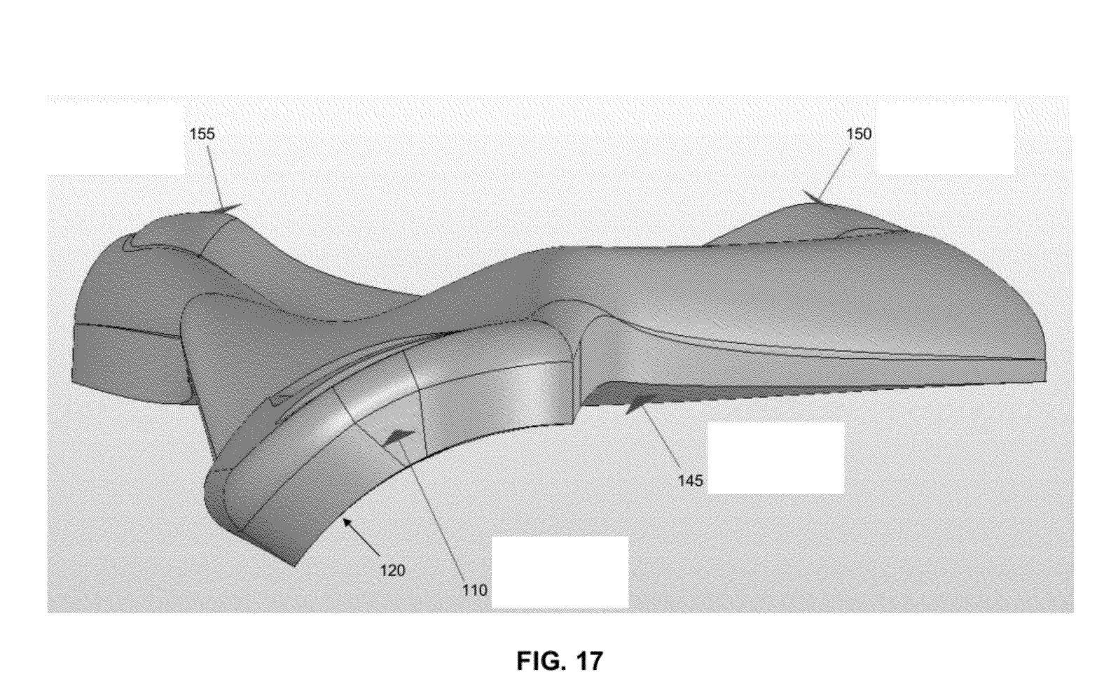

Looking next at FIGS. 13-17, there is shown a novel radius prosthesis 105 for the distal radius portion of the distal radioulnar joint. Radius prosthesis 105 generally comprises a plate 110 and a body 115. Plate 110 fixes the radius prothesis to the radius and substantially matches plate 10 of radius cutting jig 5. Body 115 provides the artificial articular surface which replaces the native sigmoid notch, as will hereinafter be discussed.

More particularly, plate 110 of radius prosthesis 105 generally comprises a concave inner bone-engaging surface 120 and a convex outer surface 125. Plate 110 of radius prosthesis 105 also comprises two or more circular screw holes 130 and a generally oblong screw hole 135. If desired, the two or more circular screw holes 130 may be of a locking type so that, when used in conjunction with locking screws, the locking screws may be fixed to the plate.

Body 115 of radius prosthesis 105 comprises a pair of inner surfaces 140, 145 which are in direct contact with the radius when radius prosthesis 105 is secured to the patient. The distal-most inner surface 140 is in contact with the portion of the distal radius remaining after removing the articular surface of the sigmoid notch (which is removed using the radius cutting jig 5 as described herein). The inner surface 145 may or may not be in contact with other portions of the radius. Body 115 terminates in a distal end surface 148. As will hereinafter be discussed, when cutting jig 5 is used to form a right angle seat in the radius, distal end surface 148 of radius prosthesis 105 may be set against that right angle seat in the excised radius so as to properly index the radius prosthesis relative to the radius (and hence relative to the ulna).

Body 115 of radius prosthesis 105 comprises an outer surface profile that generally matches the contours of the native sigmoid notch which the radius prosthesis is intended to replace. More particularly, the distal portion of prosthesis 105 comprises two crests 150, 155 and a trough 160. These elements together provide the concave articular surface of the prosthetic sigmoid notch, which comes in direct contact with counterpart portions of the ulna after joint reconstruction has been effected. It will be appreciated that the shape and size of the prosthetic sigmoid notch provided by body 115 of radius prosthesis 105 is generally matched to the shape and size of the natural sigmoid notch which is removed from the radius using radius cutting jig 5. In other words, the prosthetic sigmoid notch provided by body 115 of radius prosthesis 105 generally matches the natural sigmoid notch which is removed from the radius using radius cutting jig 5.

### Novel Cutting Jig for Preparing the Distal Ulnar Portion of the Distal Radioulnar Joint to Receive the Ulnar Prosthesis

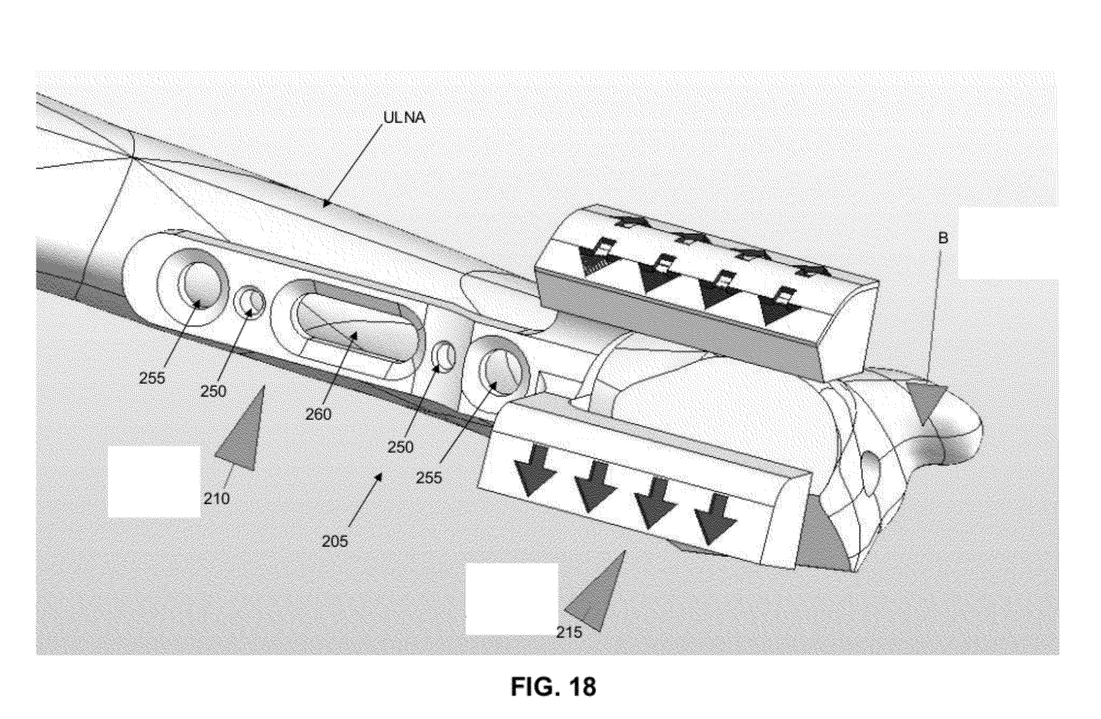

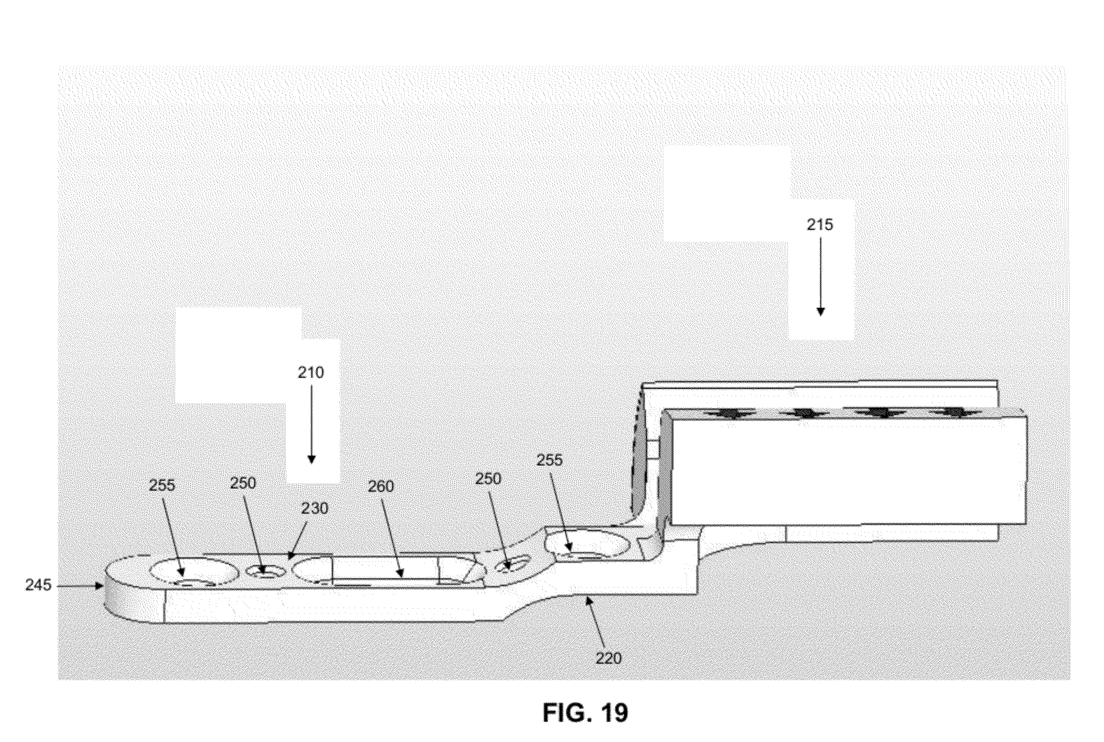

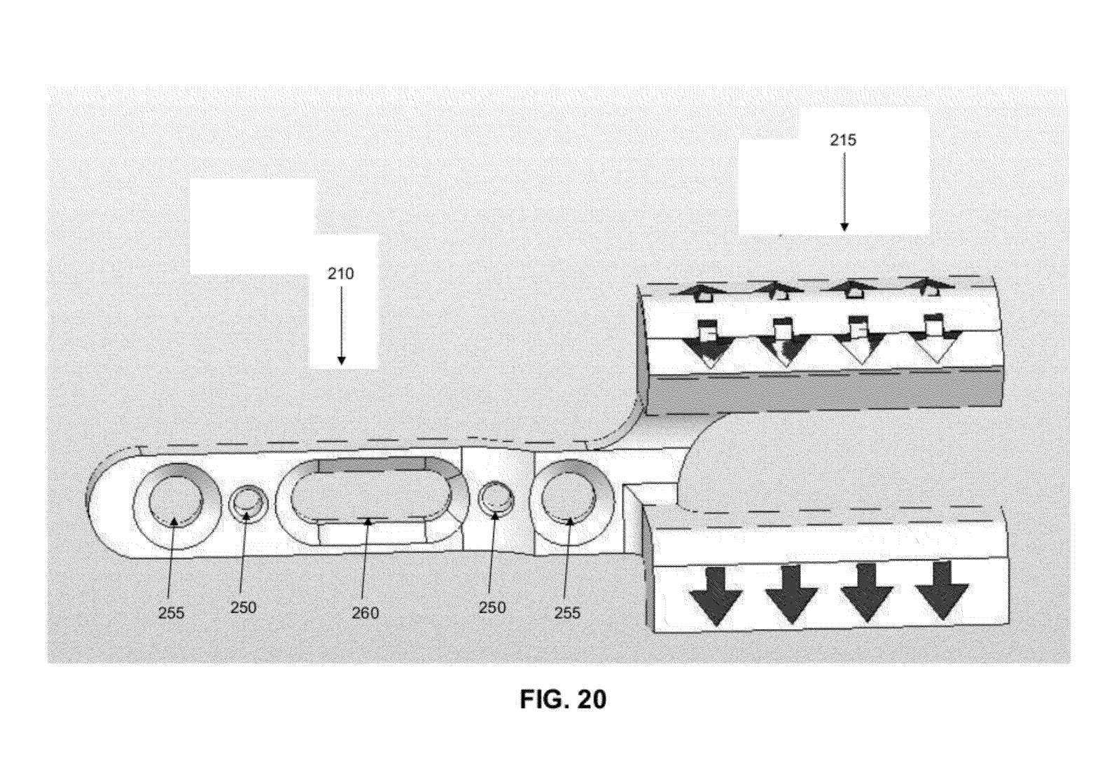

Looking next at FIGS. 18-25, there is shown a novel ulnar cutting jig 205 for preparing the distal ulnar portion of the distal radioulnar joint to receive the ulnar prosthesis. Ulnar cutting jig 205 generally comprises a plate 210 and a cutting guide 215. A neck 220 connects cutting guide 215 to plate 210.

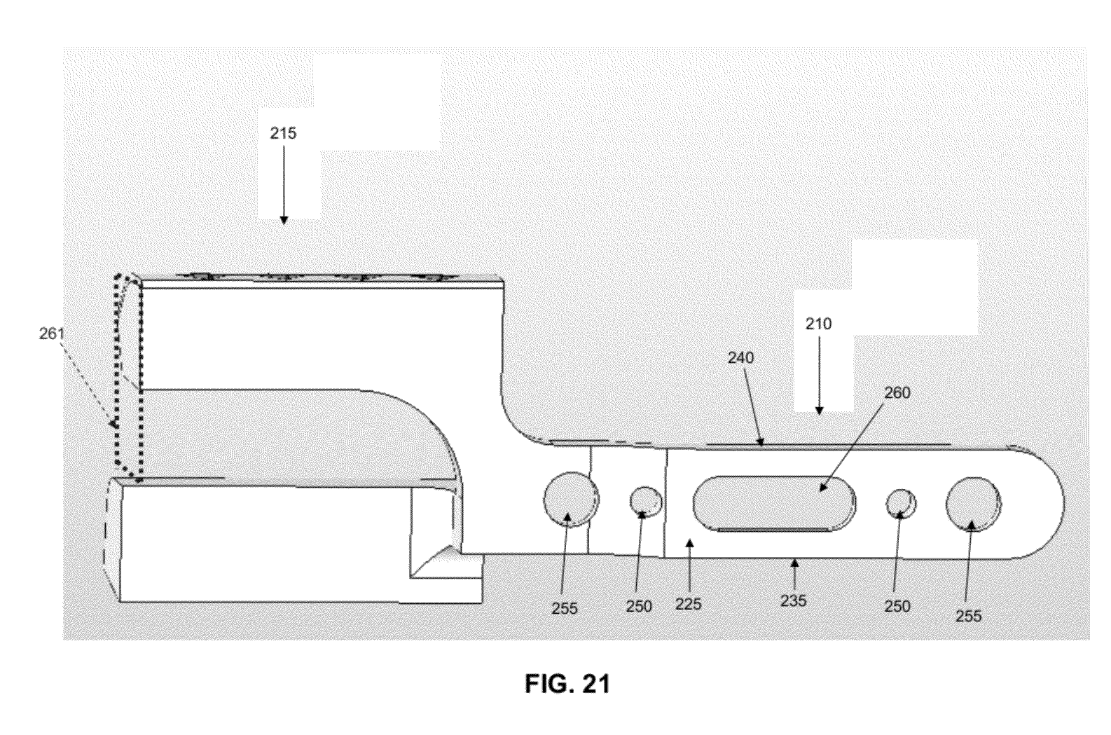

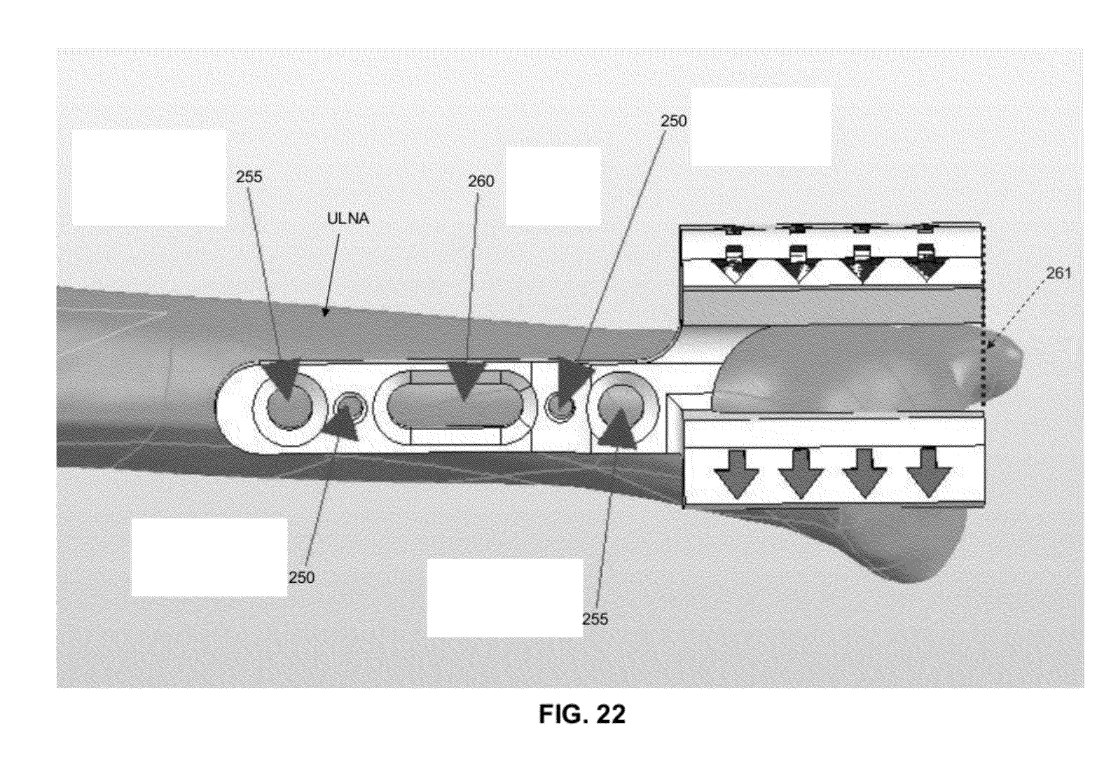

Plate 210 is configured to generally match the contours of the distal ulna and substantially matches the plate portion of the ulnar prosthesis (see below). To this end, plate 210 generally comprises an inner bone-engaging surface 225 and an outer surface 230. Plate 210 also comprises a pair of side surfaces 235, 240 and an end surface 245. In addition, plate 210 comprises two holes 250 for accommodating guidewires (see below), two or more round screw holes 255, and a generally oblong screw hole 260. If desired, a flange 261 (shown schematically in phantom in FIGS. 21 and 22, and omitted from the other figures, for clarity of illustration) may extend from one side of the ulnar cutting jig—this flange may be used to help accurately position the cutting jig on the ulna, and helps protect the TFCC from inadvertent injury by the saw blade as cuts are made in the ulna (see below).

Cutting guide 215 is designed to accept a sagittal saw or other cutting means of the sort well known in the art so as to enable a surgeon to safely and precisely excise portions of bone (e.g., the articular surface and underlying bone) at the distal end of the ulna in the region of the distal radioulnar joint (DRUJ). In one preferred form of the invention, three saw cuts are made in the ulna so as to provide three planar facets on the distal end of the ulna. These three planar facets are used to mount the ulnar prosthesis to the ulna, as will hereinafter be discussed. To this end, cutting guide 215 comprises a first guide surface 265 which may be used to cut a first facet 270 at the distal end of the ulna, a second guide surface 275 which may be used to cut a second facet 280 at the distal end of the ulna, and a third guide surface 285 which may be used to cut a third facet 290 at the distal end of the ulna.

It should be appreciated that cutting guide 215 may comprise one or more pieces. The pieces may be permanently fixed to the remainder of the cutting jig, or they may be detachable. If detachable, the pieces may be fixed to the cutting jig via a lag bolt or similar device.

### Novel Prosthesis for the Distal Ulnar Portion of the Distal Radioulnar Joint

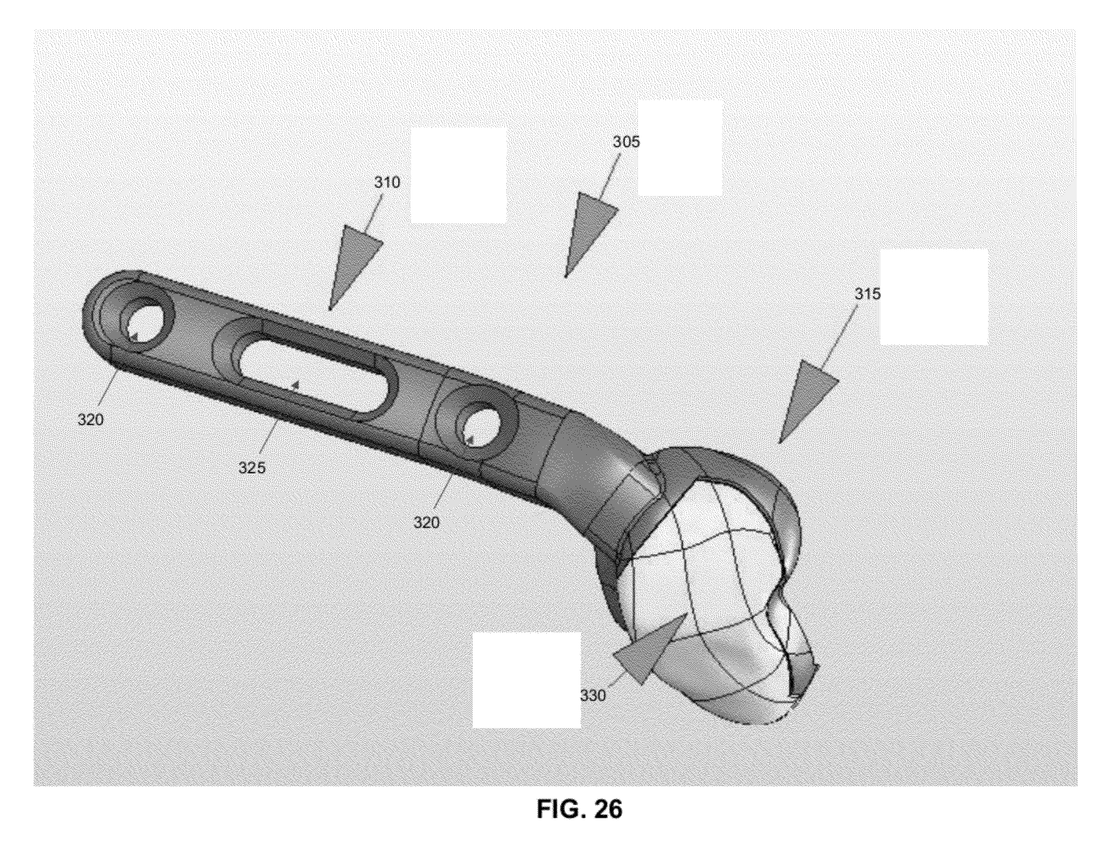

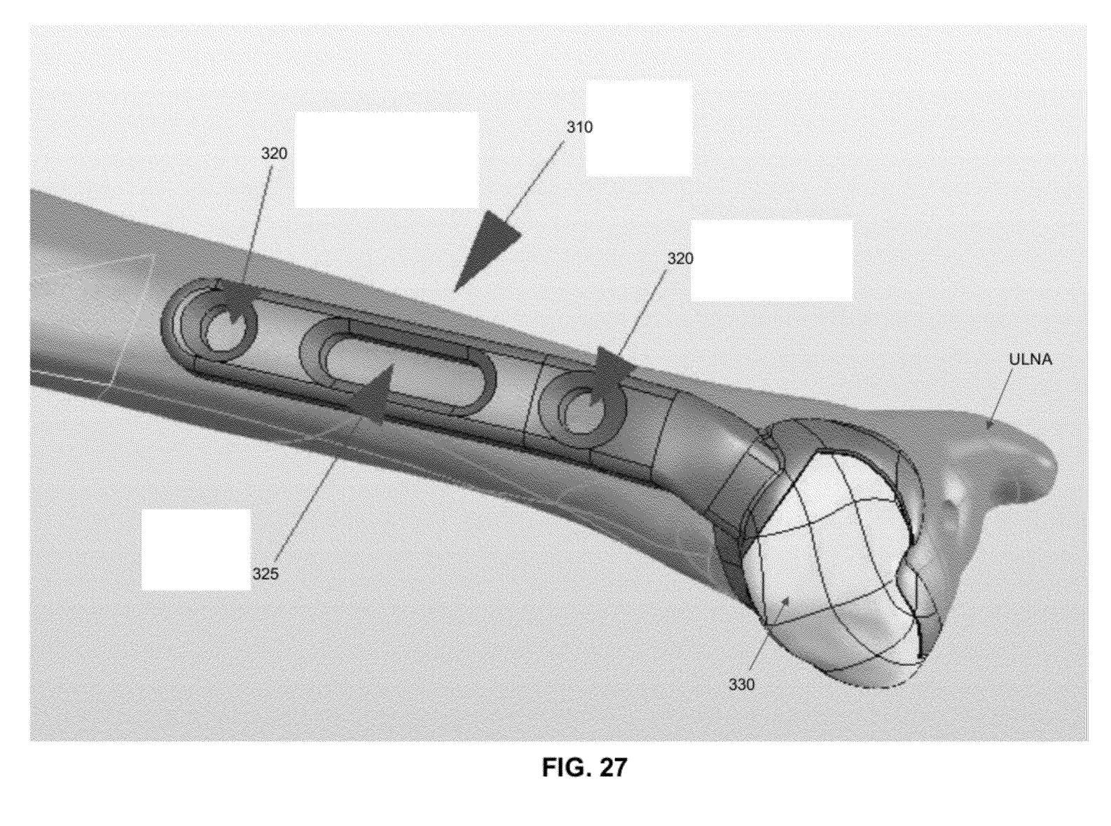

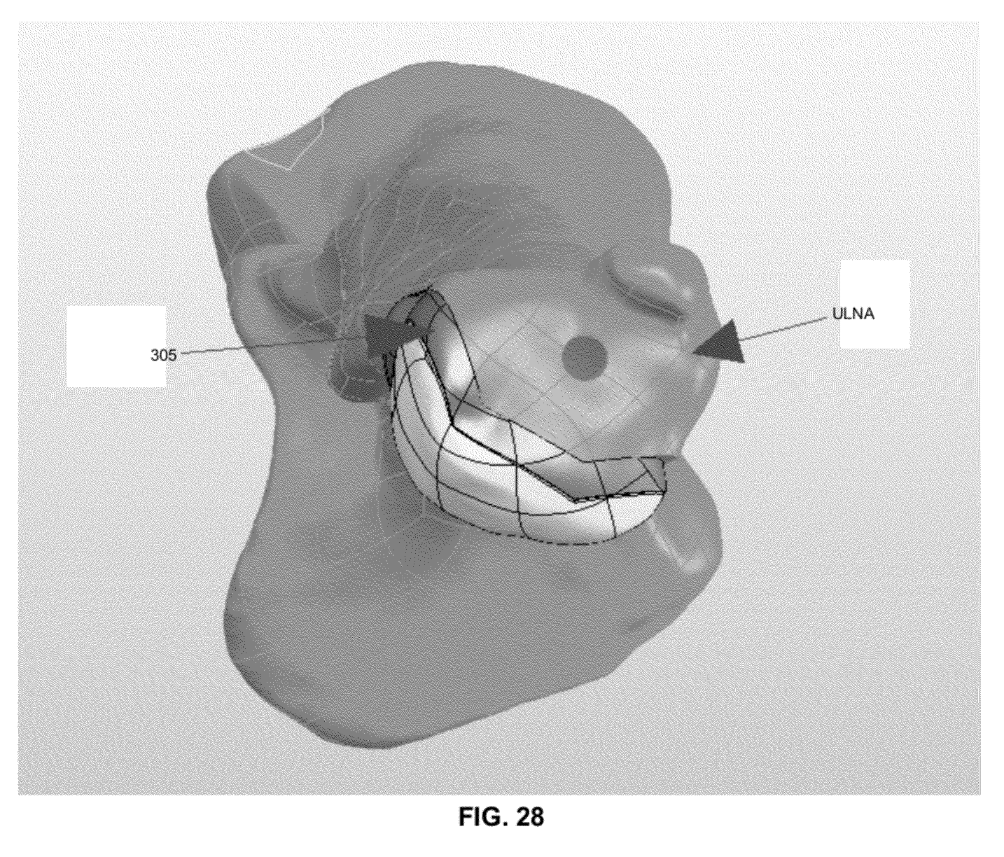

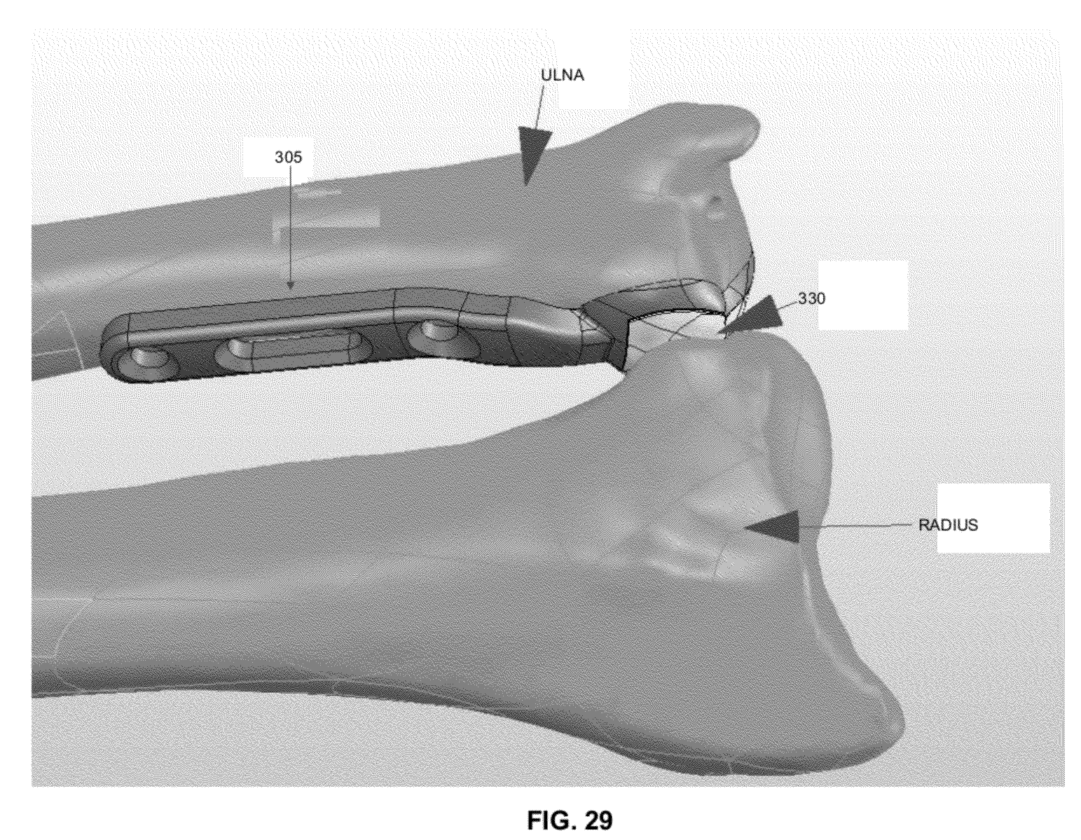

Looking next at FIGS. 26-39, there is shown a novel ulnar prosthesis 305 for the distal ulnar portion of the distal radioulnar joint. Ulnar prosthesis 305 generally comprises a plate 310 and a body 315. Plate 310 fixes the ulnar prosthesis to the ulna and generally matches plate 210 of ulnar cutting jig 205. Body 315 provides the artificial articular surface which replaces the articular surface of the native ulna.

More particularly, plate 310 comprises two or more round screw holes 320 and an oblong screw hole 325. If desired, the two or more round screw holes 320 may be of a locking type so that, when used in conjunction with locking screws, the locking screws may be fixed to the plate.

Body 315 comprises an artificial articular surface 330 which replaces the articular surface of the native ulna. To this end, artificial articular surface 330 has a convex surface profile which generally matches the surface profile of the articular surface of the native ulna.

### Total Joint Arthroplasty

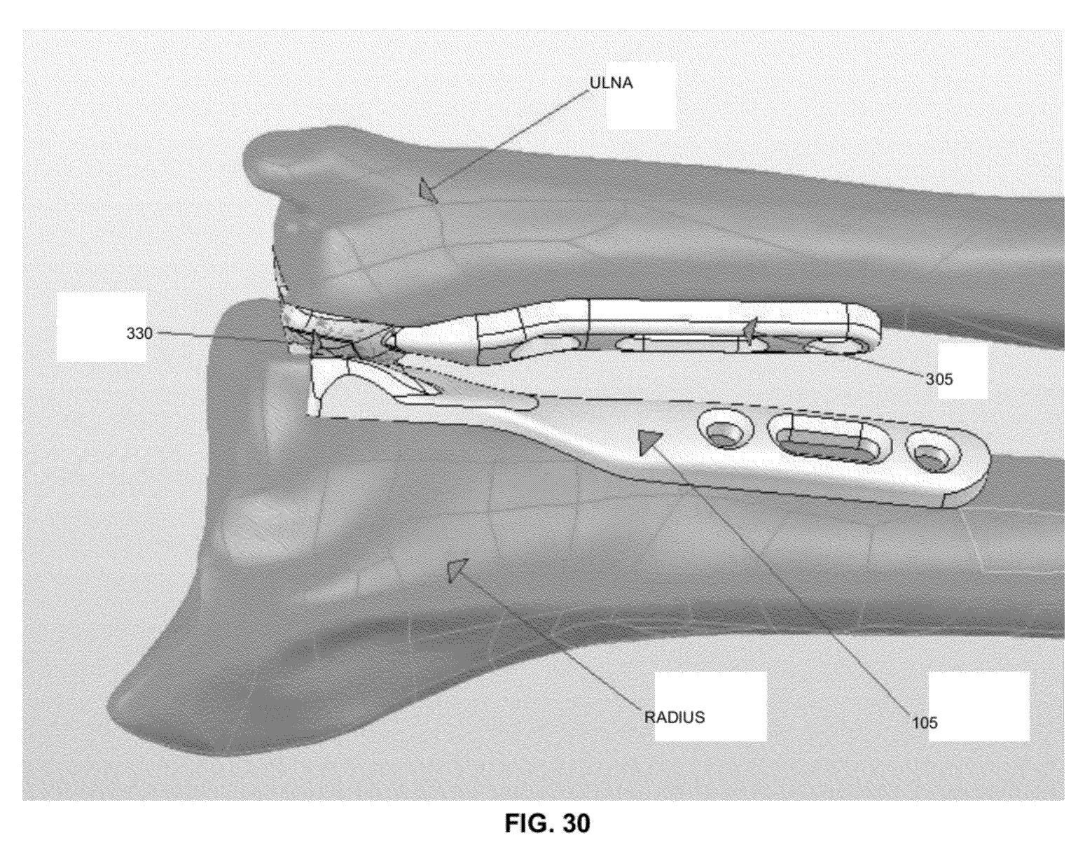

FIG. 30 shows a total joint arthroplasty effected in accordance with the present invention, with ulnar prosthesis 305 fixed to the distal end of the ulna, and radius prosthesis 105 fixed to the distal end of the radius. Note that convex articular surface 330 of ulnar prosthesis 305 seats in the concave “sigmoid notch” formed by the two crests 150, 155 and trough 160 of radius prosthesis 105.

### Alternative Construction for the Ulnar Cutting Jig

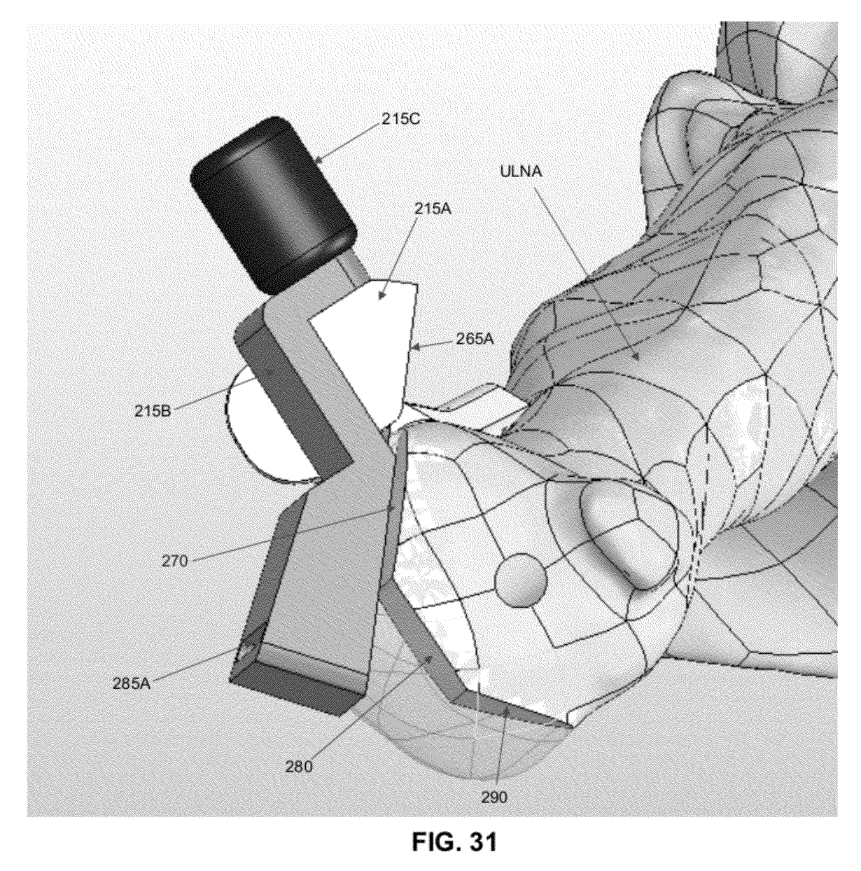

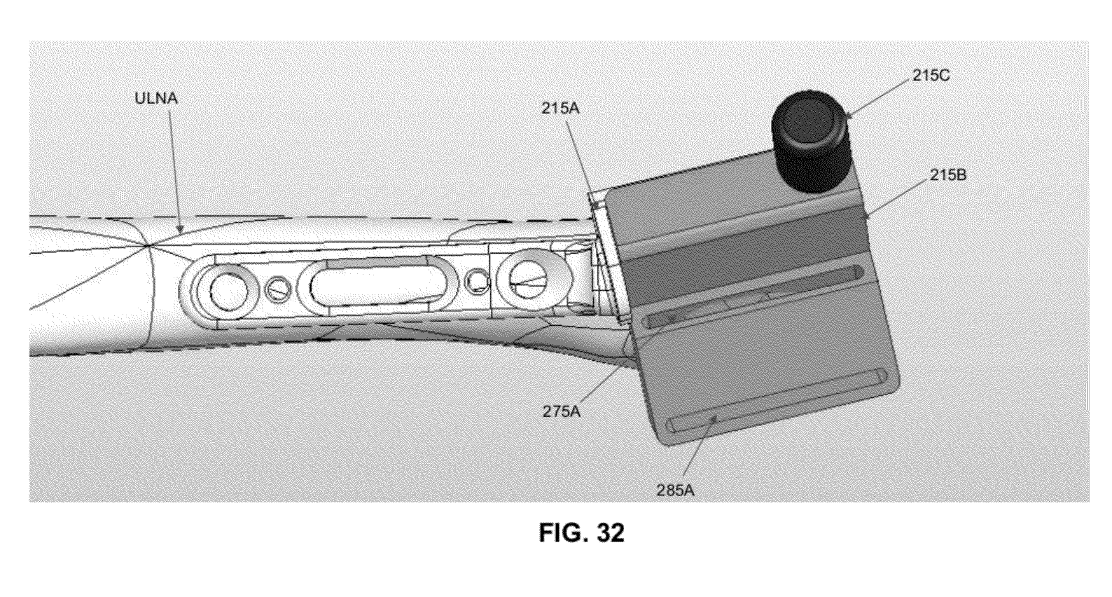

As noted above, ulnar cutting jig 205 can be fabricated out of one or more pieces. Where two or more pieces are used to form the ulnar cutting jig, these pieces may be assembled in situ. FIGS. 31 and 32 show one such construction, where cutting guide 215 comprises (i) a first element 215A carrying a first guide surface 265A for cutting first facet 270, and (ii) a second guide element 215B carrying a second guide surface 275A for cutting second facet 280 and a third guide surface 285A for cutting third facet 290. Preferably second guide element 215B is releasably mounted to first guide element 215A by a screw 215C or other similar device.

### Surgical Technique for Total Joint Arthroplasty

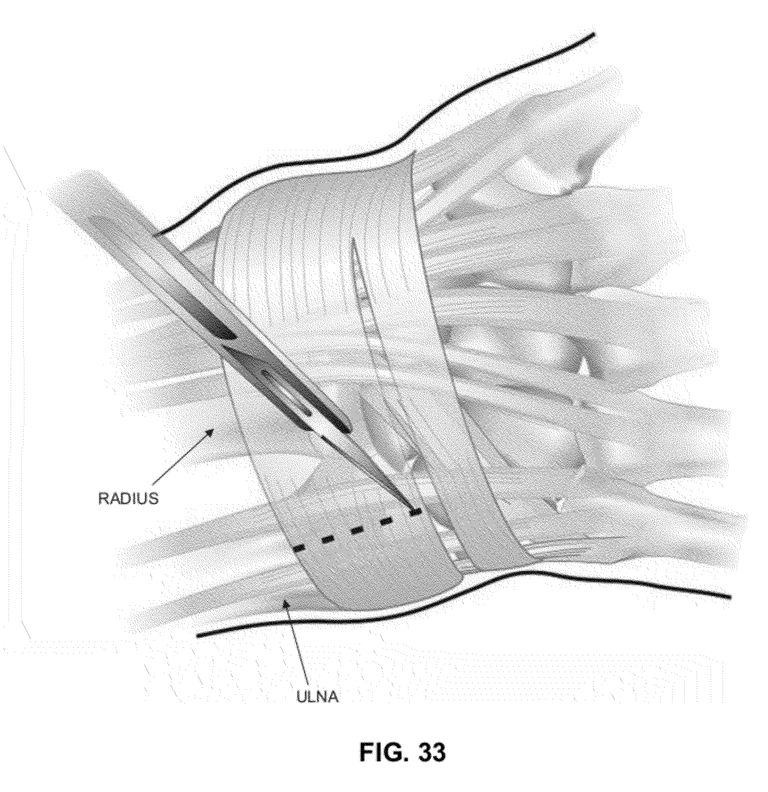

Looking next at FIGS. 33-41, a total joint arthroplasty will now be described using the method and apparatus of the present invention.

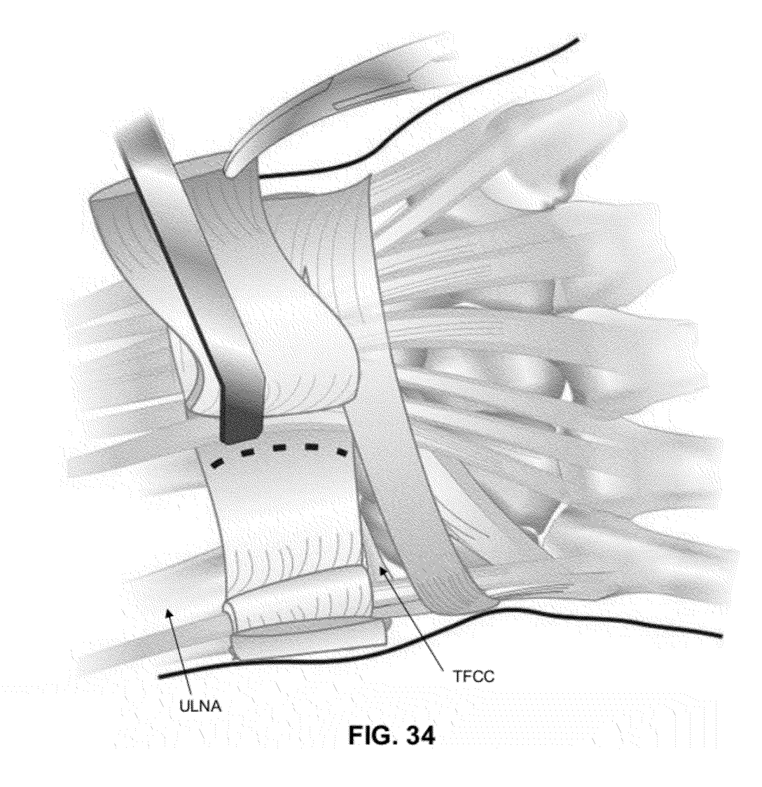

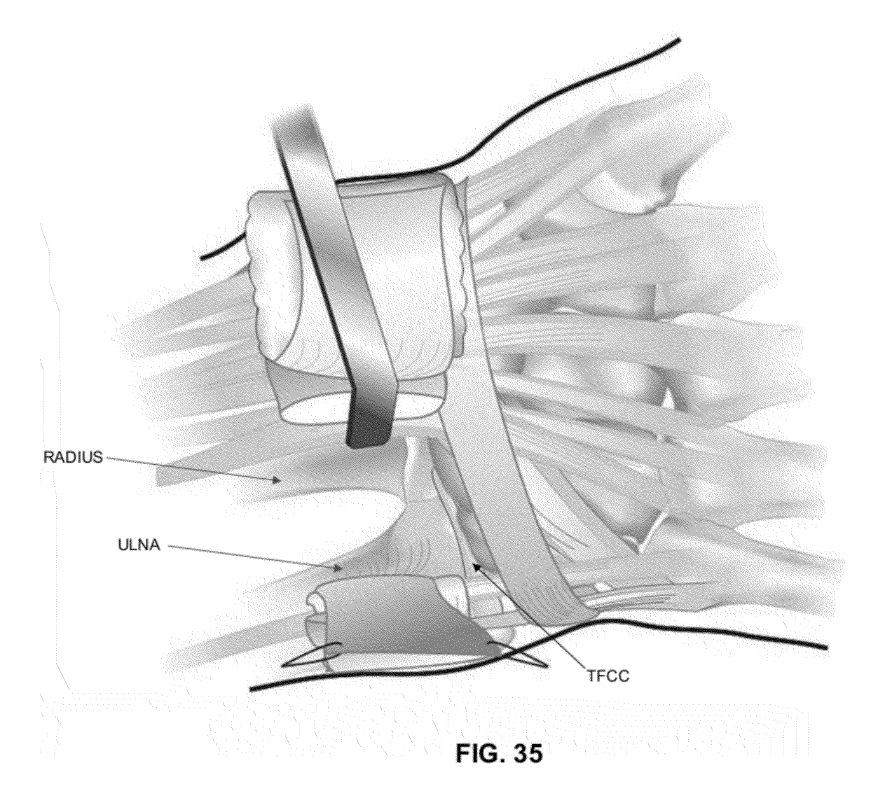

A longitudinal skin incision is made at the dorsal aspect of the wrist in line with the ring finger axis. An incision is made through the 5^{th} extensor compartment containing the Extensor Digiti Minimi (EDM) (FIG. 33). The EDM tendon and the extensor retinaculum are retracted (FIG. 34). The capsule of the DRUJ is incised in an “L” or “T” fashion with the transverse element just proximal to the TFCC. The joint articular surfaces are exposed (FIG. 35).

At this point the surgeon may address either the radius or the ulna based on preference or clinical demand. Both the radial prosthesis and the ulna prosthesis can be used singly (radial or ulnar “hemiarthroplasty”) or together (a “total joint arthroplasty”).

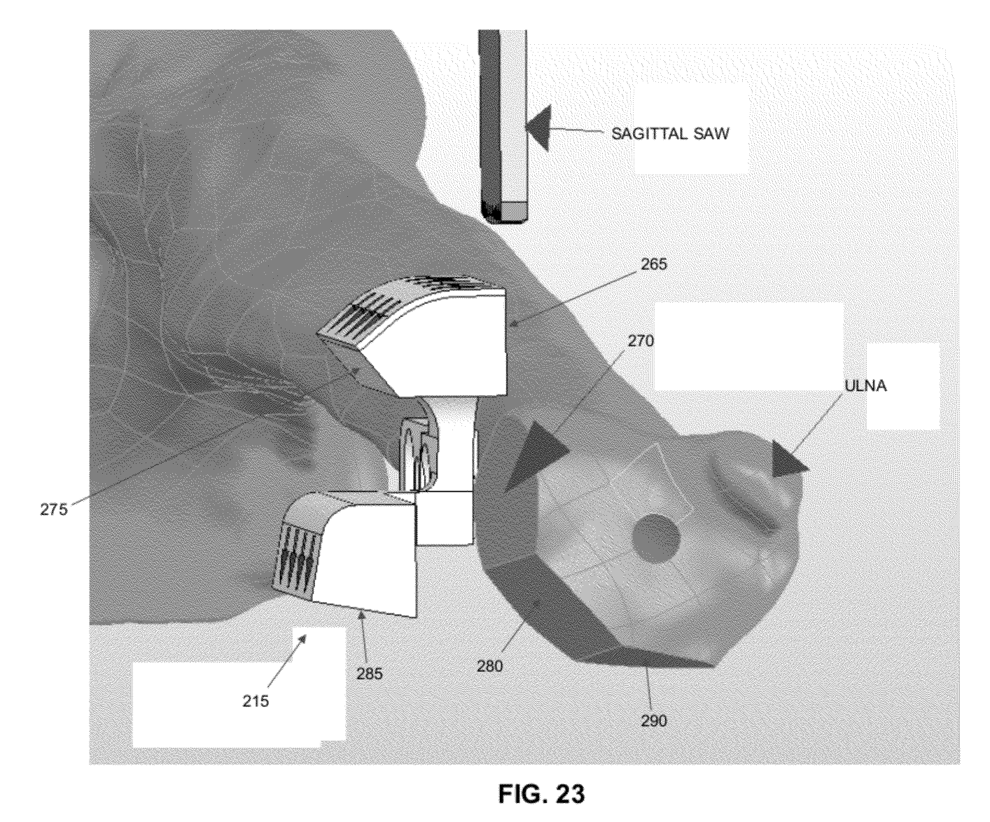

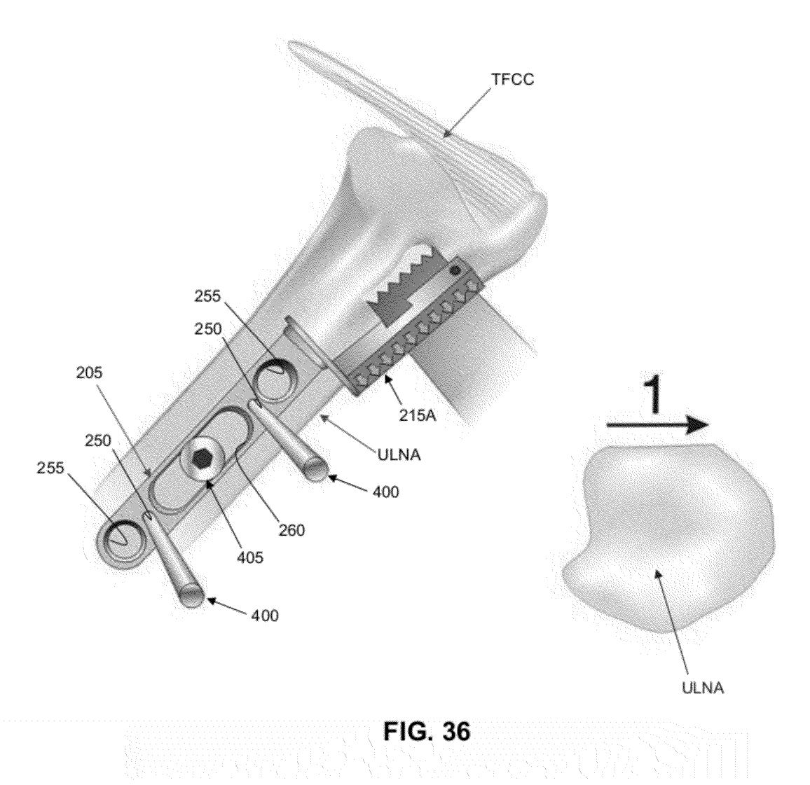

At the ulna, the shaft of the ulna is exposed proximally sufficient to allow placement of the ulna cutting jig. The 6^{th} Extensor compartment and Extensor Carpi Ulnaris (ECU) are not disturbed. The ulna cutting jig 205 is positioned with its plate portion 210 along the shaft of the ulna (FIG. 36). It is positioned just radial to the ECU and its sheath, and distally so as to be just proximal of the TFCC and dorsal distal radioulna ligaments. A flange (e.g., the aforementioned flange 261) at the distal end of the jig can facilitate proper positioning of the jig and provide protection to the TFCC. In one form of the present invention, the flange is placed at the very distal tip of the ulna and under the TFCC. Doing so will position the ulnar cutting jig so as to preserve the DRUJ ligaments/TFCC attachments. The plate portion of the ulnar cutting jig is fixed via K-wires (e.g., K-wires 400 extending through holes 250) and a non-locking screw 405 placed in the oblong hole 260 (FIG. 36). The first cut 270 is made into the ulna with a sagittal saw using surface 265/265A as a guide (FIGS. 23/31, and 36).

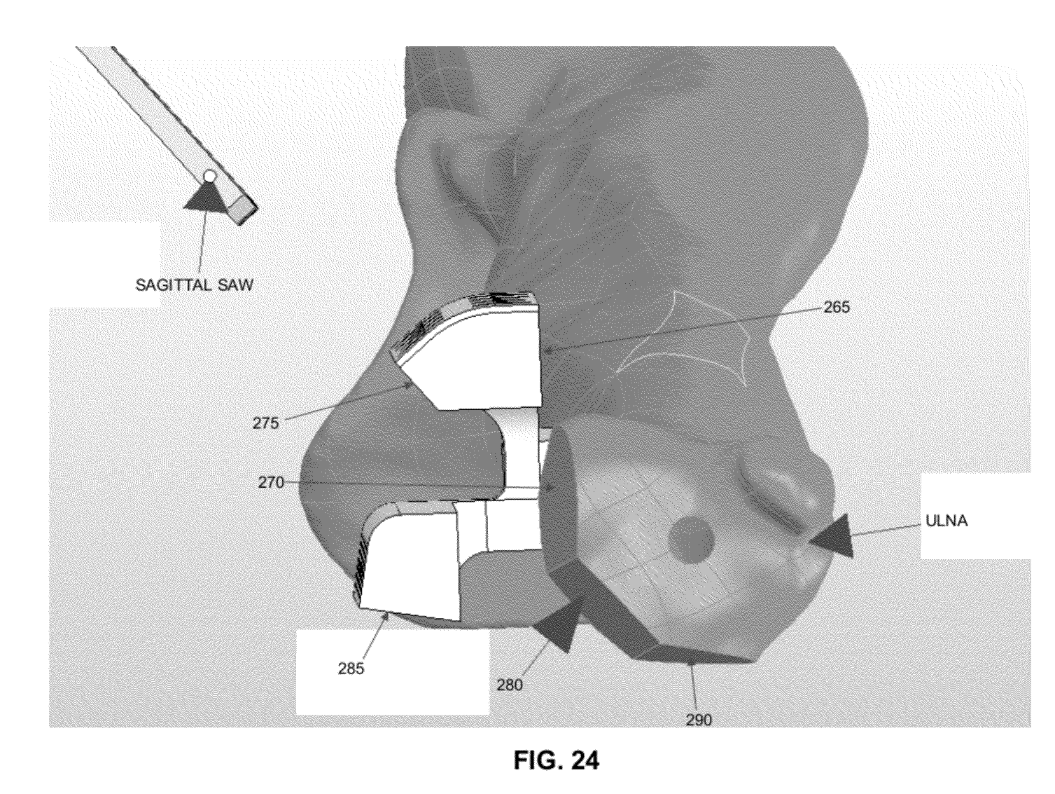

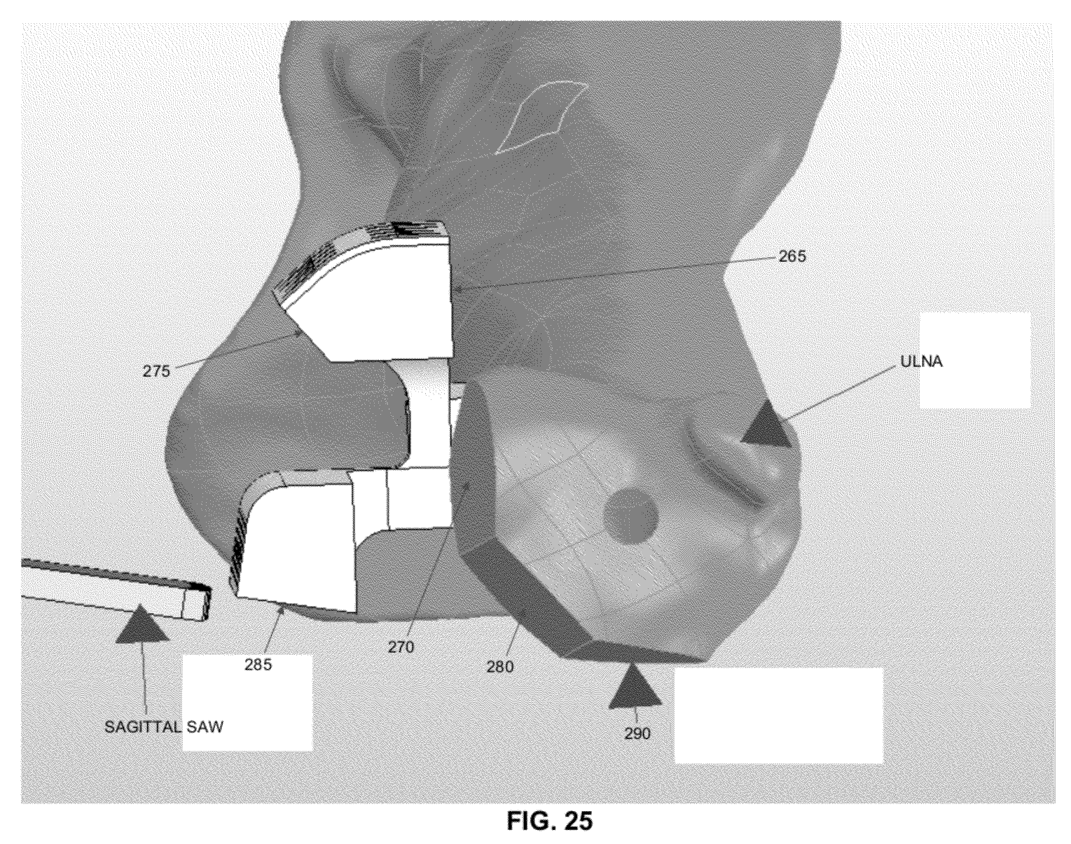

The second and third cuts 280 and 290 are then made using the corresponding guide surfaces 275/275A and 285/285A on the cutting jig (FIGS. 24 and 25/31, and 37). The K-wires, screw and cutting jig are removed upon completion of these cuts.

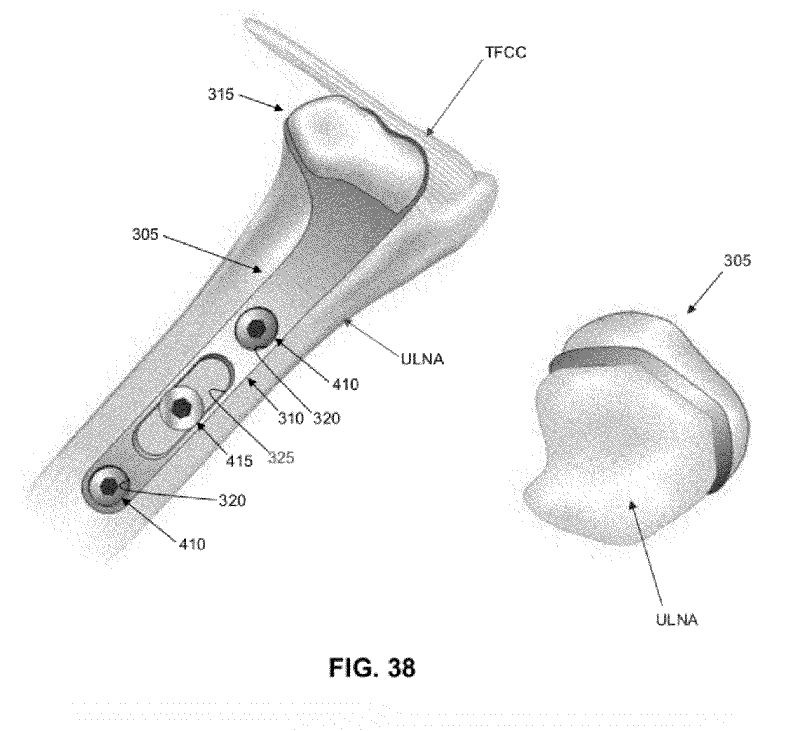

If only an ulnar hemiarthroplasty is planned, then the appropriately sized ulnar prosthesis is positioned with the plate portion 310 matching the footprint of the cutting jig's plate and the articular portion 315 resting on the 3 facet-like cuts 270, 280, 290 on the seat of the ulna (FIG. 38). The ulnar prosthesis is fixed loosely in place via a non-locking screw 415 in the oblong hole 325. This allows fine adjustment of the implant's position distal and proximal so that the distal margin of the implant is just under the TFCC and at the distal margin of the ulna. Upon final positioning, screw 415 in the oblong hole 325 is tightened. Final fixation is accomplished by placing screws 410 in the distal and proximal holes 320 (FIG. 38). In one form of the invention, screws 410 and plate holes 320 are of a locking type, so that the screws are fixed to the plate. At this point the articular surface of the ulna has been replaced with a prosthetic articular surface.

If the ulna is long in relation to the radius, the surgeon may elect to shorten the ulna. This is done in typical fashion prior to final placement of the prosthesis and after creating the 3 facet cuts (270, 280, 290) in the ulna. The ulna is cut (osteotomized) and a sufficient length of ulna bone is removed to shorten the ulna to the appropriate length. Then an ulnar prosthesis 305 with a longer plate section 310 is used, with the longer plate section 310 spanning the osteotomy and fixing the segments of the ulna in position relative to one another.

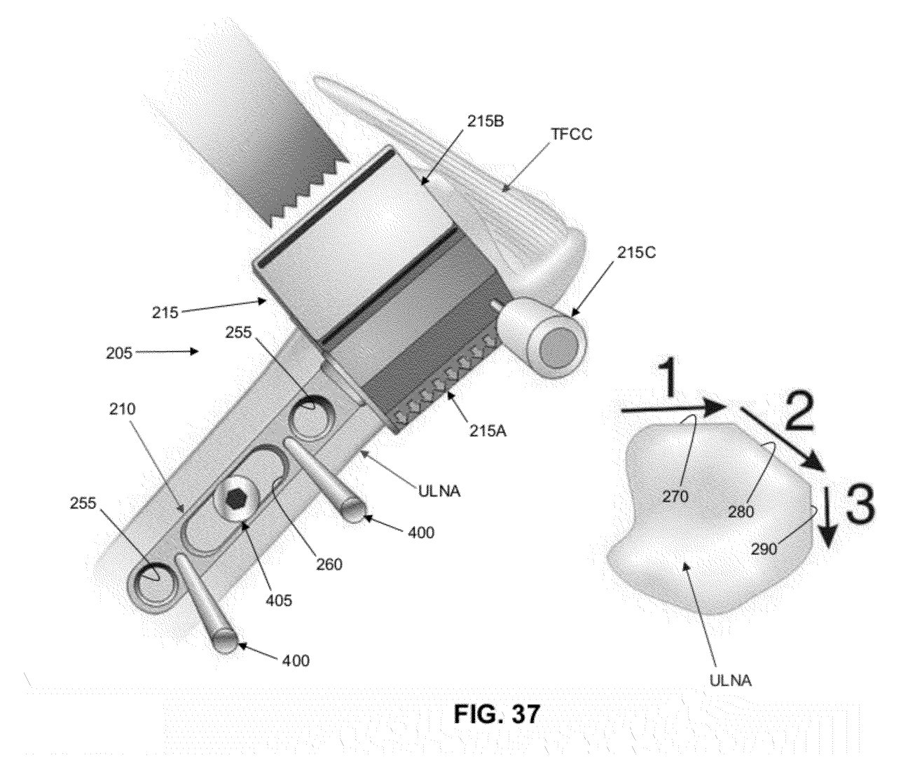

Thus it will be seen that, with an ulnar hemiarthroplasty, the cutting guide 215 on the ulnar cutting jig 205 is used to guide a saw blade or other cutting instrument so as to remove the articular surface of the distal ulna. Preferably, three facets 270, 280, 290 are cut in the ulna (FIGS. 36 and 37) using the ulnar cutting jig. Then the ulnar cutting jig 205 is removed and the ulnar prosthesis 305 is fixed in position (FIG. 38) using standard surgical implant techniques, e.g., screws 410 passing through plate holes 320 and a screw 415 passing through generally oblong screw hole 135. Note that when ulnar prosthesis 305 is fixed to the ulna, the screw 415 may occupy the hole previously occupied by the screw 405 which was used to secure the ulnar cutting jig to the ulna.

The radius is preferably addressed either before (if done alone) or after the ulnar cuts. Neither the ulnar cutting jig nor the ulnar prosthesis should be in place when addressing the radius. To approach the radius, the surgeon sharply elevates the floor of the 4^{th} extensor compartment radially from the dorsal surface of the distal radius. This may include a portion of the DRUJ joint capsule. The surgeon should take care to stay several (approximately 3) millimeters proximal to the dorsal distal margin of the radius so as to preserve the dorsal DRUJ ligaments/TFCC attachments at the lip of the radius. Additionally, the surgeon is advised to preserve the dorsal radiocarpal ligament and to avoid injury to the dorsal branch of the anterior interosseus artery. Proximal exposure requires elevation of the extensor muscle bellies from the radius and interosseous membrane.

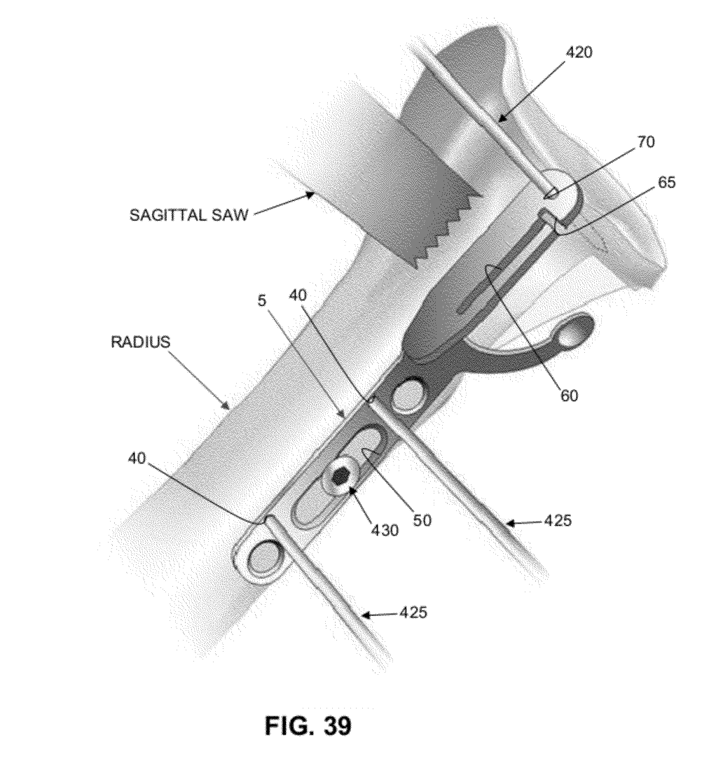

Upon completing exposure of the radius, the surgeon may insert a K-wire 420 (FIG. 39) several (approximately 3) millimeters from the dorsal lip edge of distal radius. This K-wire should be directly in the posterior-anterior plane and positioned at the subchondral apex of the lunate fossa. Doing so will position the radius cutting jig 5 so as to preserve the DRUJ ligaments/TFCC attachments and protect the lunate fossa. The radius cutting jig 5 is positioned on the distal dorsal pin (K-wire) 420 via hole 70 and secured to the dorso-ulnar cortex of the radial shaft, e.g., with K-wires 425 extending through holes 40 in plate 10.

The central oval-shaped hole 50 in plate 10 of radius cutting jig 5 may be drilled to ensure alignment when the radius prosthesis is placed. A screw 430 may be placed in the hole to provide additional stability for the cutting jig during its use. An oval-shaped hole may allow for variation of hole placement if the patient has hardware from a previous operation or requires slightly different positioning based on particular surgical findings such as ulnar length.

A saw blade or other cutting means may be inserted into the slots 60,65 in the radius cutting jig dorsally to remove a wafer-like section of the sigmoid notch (approximately 5 mm thick), leaving the distal-most ridge. Once the wafer is excised, creating a right angle seat in the radius, the radius cutting jig may be removed.

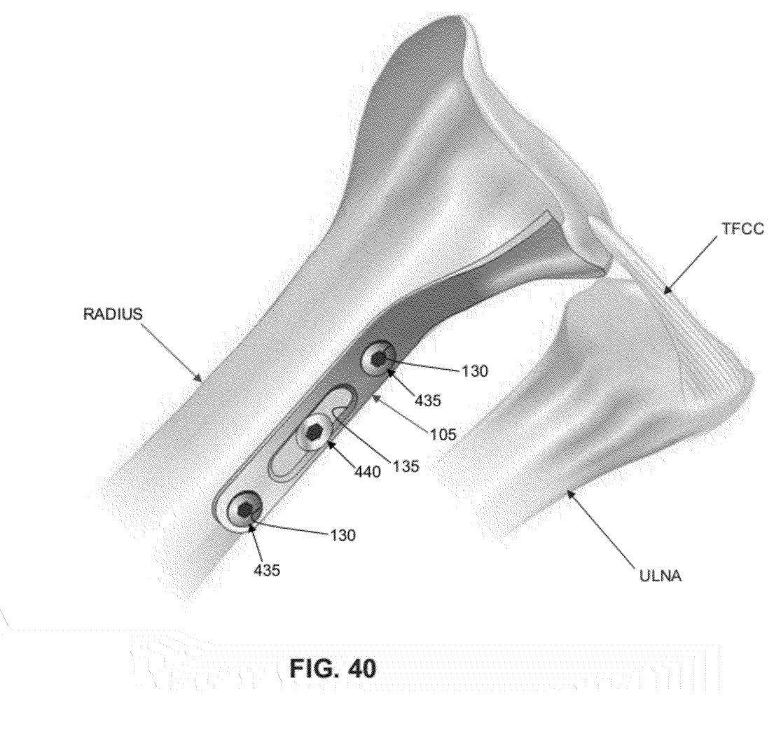

The radius prosthesis 105 may be fixed in position using standard surgical implant techniques (see FIG. 40), e.g., screws 435 passing through plate holes 130 and a screw 440 passing through generally oblong screw hole 135. In one form of the invention, screws 435 and plate holes 430 are of a locking type, so that the screws are fixed to the plate. Note that when radius prosthesis 105 is fixed to the radius, screw 440 may occupy the hole previously occupied by the screw 430 which was used to secure the radius cutting jig to the radius.

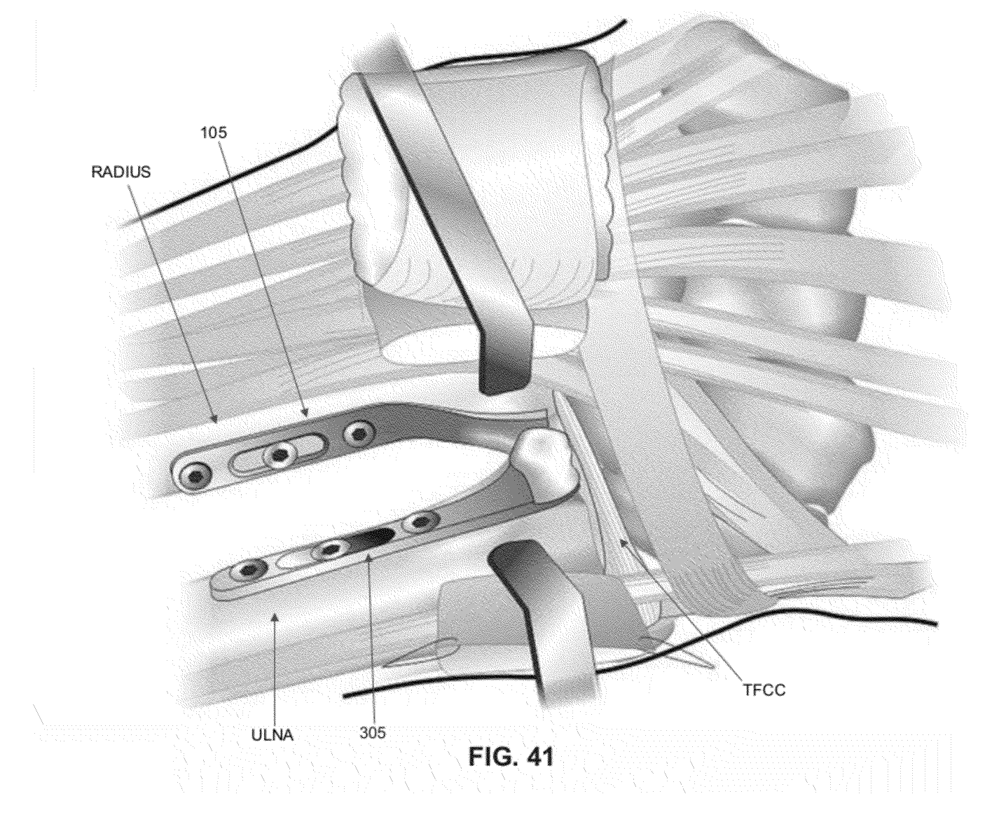

At this point a total joint arthroplasty will have been performed (FIG. 41), replacing the articular surfaces of both the ulna and the radius with prosthetic articular surfaces. It should be appreciated that this improved surgical technique closely restores or maintains normal anatomic biomechanics, maintains the option to easily revise a total joint replacement and is a straightforward approach to treating certain distal radioulnar joint afflictions.

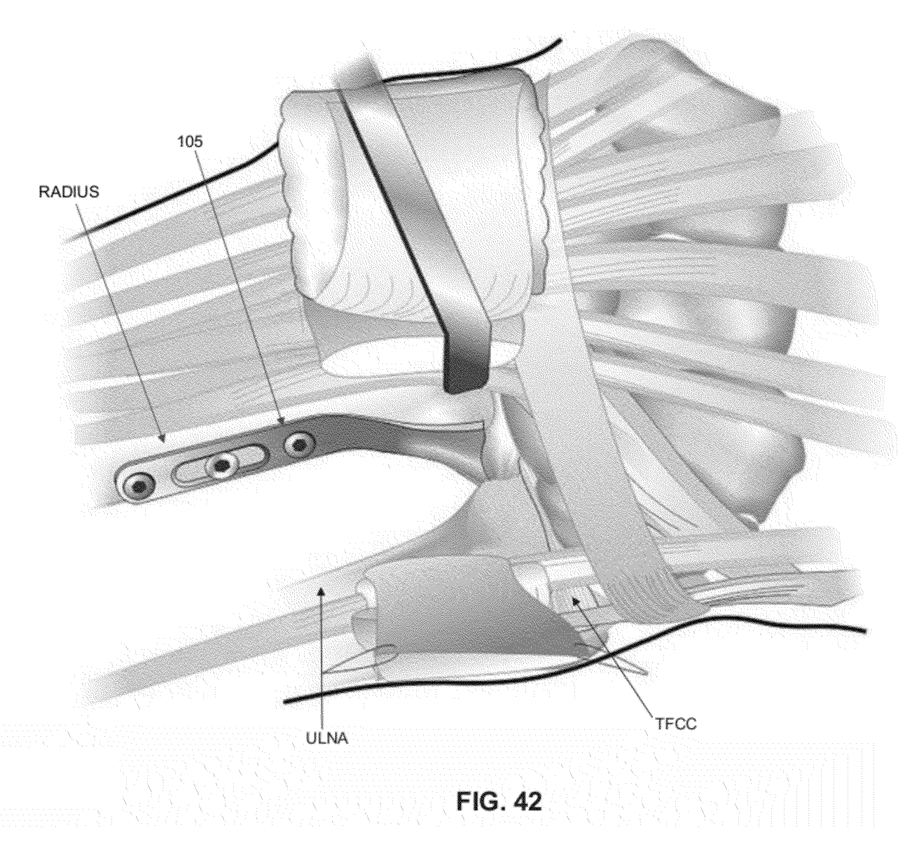

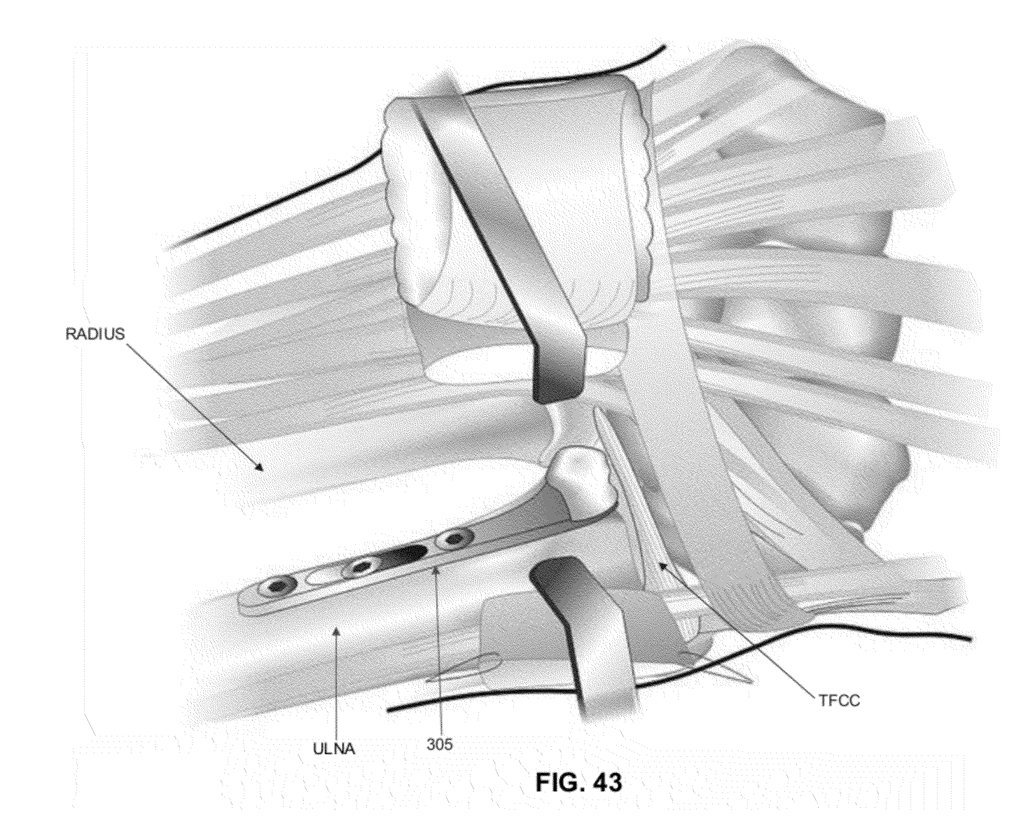

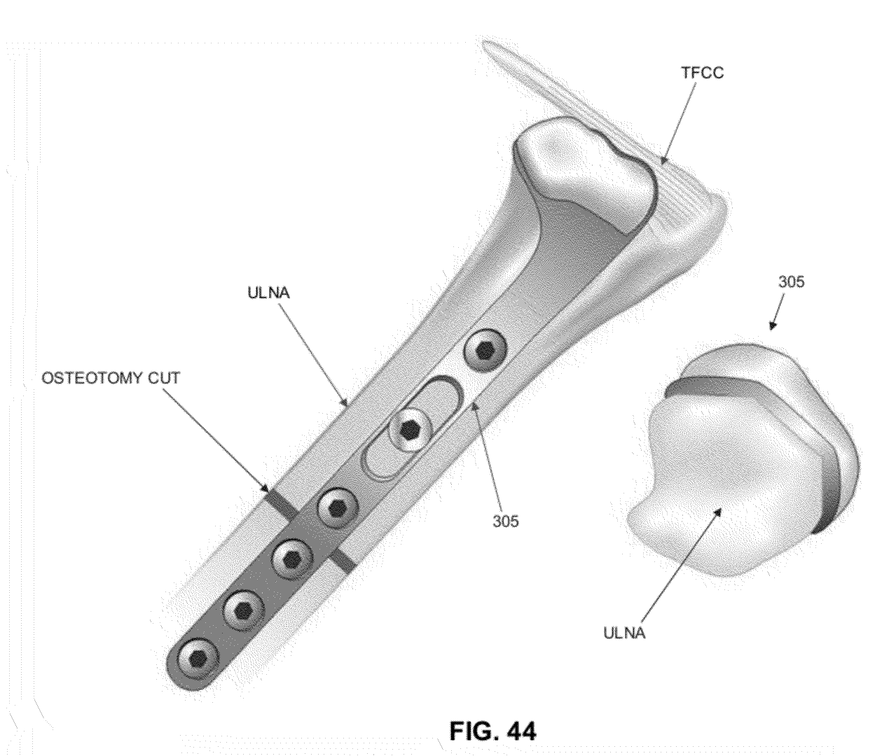

In the foregoing description, a total joint arthroplasty was performed. However, it should be appreciated that, if desired, only a radial side arthroplasty may be performed (FIG. 42) or only an ulnar side arthroplasty may be performed (FIG. 43). Furthermore, it should also be appreciated that, if desired, an osteotomy may be performed in conjunction with the ulnar side arthroplasty (FIG. 44), with the ulna prosthesis spanning the osteotomy cut.

### Use of the Novel Arthroplasty System for Other Joints

It should be appreciated that novel arthroplasty system of the present invention may also be used for reconstructing other joints in the body, e.g., a metacarpal-phalangeal joint in the hand, a trapezio-metacarpal joint in the hand (sometimes also referred to as the thumb basilar joint, or the 1st carpometacarpal joint), the great toe joint (also sometimes referred to as the 1st metatarsal-phalangeal joint), the elbow at the capitellum, the ankle, among others.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for reconstructing a joint of the sort comprising a first bone having a first articular surface and a second bone having a second articular surface, wherein the first articular surface and the second articular surface engage one another, the apparatus comprising:
a prosthesis for replacing at least a portion of the first articular surface of the first bone, the prosthesis comprising:
a prosthesis plate for positioning against an outside surface of the first bone; and
a prosthesis body connected to the prosthesis plate, the prosthesis body comprising a first prosthetic articular surface which generally matches the shape and size of the first articular surface.

2. Apparatus according to claim 1 wherein the prosthesis plate comprises an elongated structure having a longitudinal axis, and further wherein the prosthesis body is aligned with the longitudinal axis of the prosthesis plate.

3. Apparatus according to claim 1 wherein the prosthesis plate comprises at least one hole and at least one oblong opening.

4. Apparatus according to claim 1 wherein the first prosthetic articular surface comprises a concave profile.

5. Apparatus according to claim 4 wherein the prosthesis body comprises two crests connected by a trough.

6. Apparatus according to claim 4 wherein the first bone comprises the radius and the first articular surface comprises the sigmoid notch.

7. Apparatus according to claim 4 further comprising:
a cutting jig for preparing the first bone to receive the prosthesis, the cutting jig comprising:
a cutting jig plate for positioning against an outside surface of the first bone; and
a cutting jig cutting guide connected to the cutting jig plate, the cutting jig cutting guide comprising at least one longitudinal cutting slot for guiding a saw into the first bone.

8. Apparatus according to claim 7 wherein the cutting jig plate comprises at least one hole and at least one oblong opening.

9. Apparatus according to claim 7 wherein the prosthesis plate comprises at least one hole and at least one oblong opening, wherein the cutting jig plate comprises at least one hole and at least one oblong opening, and further wherein the relative positioning of the at least one hole and at least one oblong opening in the prosthesis plate matches the relative positioning of the at least one hole and at least one oblong opening in the cutting jig plate.

10. Apparatus according to claim 7 wherein the cutting jig cutting guide further comprises a transverse cutting slot.

11. Apparatus according to claim 7 further comprising a cutting jig spoon connected to the cutting jig plate.

12. Apparatus according to claim 11 wherein the cutting jig spoon comprises a concave seat for receiving a portion of the second bone.

13. Apparatus according to claim 1 wherein the first prosthetic articular surface comprises a convex profile.

14. Apparatus according to claim 13 wherein the first bone comprises the ulna and the first articular surface comprises the head of the ulna.

15. Apparatus according to claim 13 further comprising:
a cutting jig for preparing the first bone to receive the prosthesis, the cutting jig comprising:
a cutting jig plate for positioning against an outside surface of the first bone; and
a cutting jig cutting guide connected to the cutting jig plate, the cutting jig cutting guide comprising a first guide surface for use in cutting a first facet on the first bone, a second guide surface for use in cutting a second facet on the first bone, and a third guide surface for use in cutting a third facet on the first bone.

16. Apparatus according to claim 15 wherein the cutting jig plate comprises at least one hole and at least one oblong opening.

17. Apparatus according to claim 15 wherein the prosthesis plate comprises at least one hole and at least one oblong opening, wherein the cutting jig plate comprises at least one hole and at least one oblong opening, and further wherein the relative positioning of the at least one hole and at least one oblong opening in the prosthesis plate matches the relative positioning of the at least one hole and at least one oblong opening in the cutting jig plate.

18. Apparatus according to claim 15 wherein the cutting jig cutting guide comprises a single piece.

19. Apparatus according to claim 15 wherein the cutting jig cutting guide comprises two pieces, and further wherein the first guide surface is formed on a first one of the two pieces and the second and third guide surfaces are formed on the second one of the two pieces.

20. Apparatus for reconstructing a joint of the sort comprising a first bone having a first articular surface and a second bone having a second articular surface, wherein the first articular surface and the second articular surface engage one another, the apparatus comprising:
a cutting jig for preparing the first bone to receive a prosthesis, the cutting jig comprising:
a plate for positioning against the outside surface of the first bone; and
a cutting guide connected to the plate, the cutting guide comprising at least one longitudinal cutting slot.

21. Apparatus according to claim 20 wherein the first bone comprises the radius and the first articular surface comprises the sigmoid notch.

22. Apparatus for reconstructing a joint of the sort comprising a first bone having a first articular surface and a second bone having a second articular surface, wherein the first articular surface and the second articular surface engage one another, the apparatus comprising:
a cutting jig for preparing the first bone to receive a prosthesis, the cutting jig comprising:
a plate for positioning against the outside surface of the first bone; and
a cutting guide connected to the plate, the cutting guide comprising a first guide surface for use in cutting a first facet on the first bone, a second guide surface for use in cutting a second facet on the first bone, and a third guide surface for use in cutting a third facet on the first bone.

23. Apparatus according to claim 22 wherein the first bone comprises the ulna and the first articular surface comprises the head of the ulna.

24. A method for reconstructing a joint of the sort comprising a first bone having a first articular surface and a second bone having a second articular surface, wherein the first articular surface and the second articular surface engage one another, the method comprising:
providing a prosthesis for replacing at least a portion of the first articular surface of the first bone, the prosthesis comprising:
a prosthesis plate for positioning against an outside surface of the first bone; and
a prosthesis body connected to the prosthesis plate, the prosthesis body comprising a first prosthetic articular surface which generally matches the shape and size of the first articular surface; and
securing the prosthesis to the first bone so that the first prosthetic articular surface is positioned in place of the first articular surface.

25. A method according to claim 24 wherein the prosthesis plate comprises an elongated structure having a longitudinal axis, and further wherein the prosthesis body is aligned with the longitudinal axis of the prosthesis plate.

26. A method according to claim 24 wherein the first prosthetic articular surface comprises a concave profile.

27. A method according to claim 24 wherein the first bone comprises the radius and the first articular surface comprises the sigmoid notch.

28. A method according to claim 24 further comprising:
providing a cutting jig for preparing the first bone to receive the prosthesis, the cutting jig comprising:
a cutting jig plate for positioning against an outside surface of the first bone; and
a cutting jig cutting guide connected to the cutting jig plate, the cutting jig cutting guide comprising at least one longitudinal cutting slot for guiding a saw into the first bone; and
using the cutting jig to prepare the first bone to receive the prosthesis.

29. A method according to claim 28 wherein the prosthesis plate comprises at least one hole and at least one oblong opening, wherein the cutting jig plate comprises at least one hole and at least one oblong opening, and further wherein the relative positioning of the at least one hole and at least one oblong opening in the prosthesis plate matches the relative positioning of the at least one hole and at least one oblong opening in the cutting jig plate.

30. A method according to claim 28 wherein the cutting jig cutting guide further comprises a transverse cutting slot.

31. A method according to claim 28 further comprising a cutting jig spoon connected to the cutting jig plate.

32. A method according to claim 24 wherein the first prosthetic articular surface comprises a convex profile.

33. A method according to claim 24 wherein the first bone comprises the ulna and the first articular surface comprises the head of the ulna.

34. A method according to claim 24 further comprising:
providing a cutting jig for preparing the first bone to receive the prosthesis, the cutting jig comprising:
a cutting jig plate for positioning against an outside surface of the first bone; and
a cutting jig cutting guide connected to the cutting jig plate, the cutting jig cutting guide comprising a first guide surface for use in cutting a first facet on the first bone, a second guide surface for use in cutting a second facet on the first bone, and a third guide surface for use in cutting a third facet on the first bone; and
using the cutting jig to prepare the first bone to receive the prosthesis.

35. A method according to claim 34 wherein the prosthesis plate comprises at least one hole and at least one oblong opening, wherein the cutting jig plate comprises at least one hole and at least one oblong opening, and further wherein the relative positioning of the at least one hole and at least one oblong opening in the prosthesis plate matches the relative positioning of the at least one hole and at least one oblong opening in the cutting jig plate.

36. A method according to claim 34 wherein the cutting jig cutting guide comprises a single piece.

37. A method according to claim 34 wherein the cutting jig cutting guide comprises two pieces, and further wherein the first guide surface is formed on a first one of the two pieces and the second and third guide surfaces are formed on the second one of the two pieces.
